(19) United States
(12) Patent Application Publication
(10) Pub. No.: US 2018/0221457 A1
(43) Pub. Date: Aug. 9, 2018

(54) GLYCEROL-3-PHOSPHATE PHOSPHATASE ACTIVATORS

(71) Applicant: VAL-CHUM, LIMITED PARTNERSHIP, Montréal (CA)

(72) Inventors: Murthy S.R. Madiraju, Brossard (CA); Marc Prentki, Ville Mont-Royal (CA); Yves Mugabo, Repentigny (CA)

(21) Appl. No.: 15/749,202

(22) PCT Filed: Jul. 28, 2016
(86) PCT No.: PCT/CA2016/050885

§ 371 Date: Jan. 31, 2018

Related U.S. Application Data
(60) Provisional application No. 62/199,259, filed on Jul. 31, 2015.

(51) Int. Cl. A61K 38/46; A61P 35/00; G01N 33/574; A61P 3/10

(52) U.S. Cl. CPC A61K 38/465; A61P 35/00; G01N 2800/042; A61P 3/10; G01N 2800/044; G01N 33/57438

(57) ABSTRACT

The present disclosure relates to the identification and the use of activators of a mammalian glycerol-3-phosphate phosphatase (hG3PP) for increasing Gro3P conversion to glycerol and glycerol release from a mammalian cell. The activators of hG3PP can be used in the prevention, treatment and/or alleviation of symptoms associated with obesity, type 2 diabetes and/or metabolic syndrome X. The activators of hG3PP can be used in the prevention, treatment and/or alleviation of symptoms associated with cancer.

CROSS-REFERENCE TO RELATED APPLICATIONS AND DOCUMENTS

This application claims priority from U.S. provisional patent application 62/199259 filed on Jul. 31, 2015. This application also contains a sequence listing which has been concurrently filed herewith. The content of the priority and of the sequence listing is incorporated herewith in its entirety.

### TECHNOLOGICAL FIELD

This disclosure relates to the relationship between the activity of a glycerol-3-phosphate phosphatase (herein referred to as “G3PP”) and glycerol formation/release in mammalian cells. The present disclosure provides screening assays to identify agents capable of activating G3PP as well as gene therapy tools for increasing the activity of G3PP.

### BACKGROUND

The glycerolipid/fatty acid (GL/FA) cycle, which is central to energy homeostasis, balances glucose and lipid metabolism and generates metabolic signals. This cycle is deregulated in obesity and type 2 diabetes (T2D). Under conditions of fuel surfeit with excessive glucose and free fatty acid (FFA) supply, a substantial portion of glucose is utilized in mammalian cells via formation of glycerol-3-phosphate (Gro3P) and its incorporation into GL via GL/FA cycle. The cycle consists of lipogenesis and lipolysis segments and generates intermediates for the synthesis of various types of complex lipids but also signals that control many biological processes, including insulin secretion and action. The proper operation of this cycle possibly protects β-cells and other cell types from glucolipotoxicity and metabolic stress.

Lipogenesis, i.e., the successive esterification of glycolysis-derived Gro3P with fatty acyl-CoA (FA-CoA), produces triglyceride (TG), which can be stored as lipid droplets. Lipolysis of TG is initiated by adipose triglyceride lipase, to generate diacylglycerol (DAG), which is hydrolyzed by hormone sensitive lipase to give rise to monoacylglycerol (MAG). MAG hydrolysis either by classical MAG lipase or by α/β-Hydrolase domain-6 (ABHD6) to glycerol and FFA completes the lipolytic segment of the GL/FA cycle.

It would be highly desirable to be provided with a therapeutic target for screening agents involved in the regulation of glucose and lipid metabolism as well as in the response to metabolic stress. It would also be desirable to be provided with modulators of such therapeutic target for providing therapeutic benefits in the metabolism of glucose and lipids as well as in the response to metabolic stress.

### BRIEF SUMMARY

The present disclosure concerns the activity of the human glycerol-3-phosphate phosphatase (herein referred to as hG3PP) and its biological effects in the metabolism of glucose and lipids as well as in the response to metabolic stress. As it is described herein, when the activity of the hG3PP is increased, more intracellular glycerol is produced from glycerol-3-phosphate and more glycerol is released from mammalian cells (such as human cells). In return, this increase in glycerol production and release limit or impede gluconeogenesis which can be especially useful for the treatment of conditions in which gluconeogenesis is elevated, such as, for example, type II diabetes, obesity, metabolic syndrome X and/or cancer.

In a first aspect, the present disclosure provides a method for characterizing the usefulness of a test agent to increase glycerol production and glycerol release from a mammalian cell. Broadly, the method comprises : (a) providing a human glycerol-3-phosphate phosphatase (hG3PP) and a substrate of the human glycerol-3-phosphate phosphatase that can be cleaved by the hG3PP to generate at least one detectable moiety; (b) combining the test agent with the hG3PP and the substrate under conditions so as to allow the cleavage of the substrate by the hG3PP and the generation of the at least one detectable moiety; (c) determining a test amount of the at least one detectable moiety generated at step (b); (d) comparing the test amount with a first control amount of the at least one detectable moiety, wherein the first control amount is derived from or obtained by combining the hG3PP and the substrate, in the absence of the test agent, under conditions so as to allow the cleavage of the substrate by the hG3PP and the generation of the at least one detectable moiety; and (e) characterizing the test agent as being useful for increasing glycerol production and glycerol release from the mammalian cell when the test amount is determined to be higher than the first control amount. In an embodiment, at step (a), the substrate (which can be, for example glycerol-3-phosphate) is provided at or near a saturating concentration. In another embodiment, the at least one detectable moiety can be glycerol or inorganic phosphate. In still another embodiment, step (a) further comprises providing a cellular extract of the mammalian cell comprising the hG3PP, providing the hG3PP in a substantially isolated form and/or providing the substrate in a substantially isolated form. In yet another embodiment, the method further comprises (f) providing a second control amount obtained by combining the hG3PP, the substrate and succinic acid or a succinate salt under conditions so as to allow the cleavage of the substrate by the hG3PP and the generation of the at least one detectable moiety; (g) comparing the second control amount with the first control amount; and (h) characterizing the agent as being useful for increasing glycerol production and glycerol release from the mammalian cell from the mammalian cell only when the second control amount is higher than the first control amount. In yet another embodiment, the mammalian cell is a mammalian pancreatic β cell. In such embodiment, the method can further comprise characterizing the test agent useful for increasing glycerol production and glycerol release from the mammalian pancreatic β cell as being useful, in the mammalian pancreatic β cell, for decreasing glucose-stimulated insulin secretion, for decreasing glucotoxicity, for decreasing glucolipotoxicity, for decreasing diacylglycerol synthesis, for decreasing triglyceride synthesis, for decreasing phospholipid synthesis, for decreasing lysophosphatidic acid synthesis, for decreasing oxygen consumption, for decreasing ATP production and/or for increasing free fatty acid release. In yet another embodiment, the mammalian cell is a mammalian hepatocyte. In such embodiment, the method can further comprises characterizing the test agent useful for increasing glycerol production and glycerol release from the mammalian hepatocyte as being useful, in the mammalian hepatocyte, for decreasing glucose synthesis, for decreasing lactate synthesis, for decreasing lactate release, for decreasing diacylglycerol synthesis, for decreasing triglyceride synthesis and/or for increasing free fatty acid oxidation. In still a further embodiment, the mammalian cell is a cancerous cell. In such embodiment, the method can further comprise characterizing the test agent useful for limiting the viability and/or the proliferation of the cancerous cell when the test amount is determined to be higher than the first control amount. In still another embodiment, the method can further comprise characterizing the test agent useful for increasing glycerol production and glycerol release from the mammalian cell as being useful for the prevention, treatment and/or the alleviation of symptoms associated with obesity, type 2 diabetes and/or metabolic syndrome X in a mammalian subject, when the test amount is determined to be higher than the first control amount. In yet another embodiment, the method can further comprise characterizing the test agent useful for increasing glycerol production and glycerol release from the mammalian cell as being useful for the prevention, treatment and/or the alleviation of symptoms associated with a cancer in a mammalian subject, when the test amount is determined to be higher than the first control amount.

In a second aspect, the present disclosures provides a method for increasing glycerol production and glycerol release from a mammalian cell. Broadly, the method comprises contacting an effective amount of an agent capable of increasing the biological activity of a human glycerol-3-phosphate phosphatase (hG3PP) in the mammalian cell. The agent can be, for example, is succinic acid, a succinate salt and/or a nucleic acid expression system encoding the hG3PP. In an embodiment, the mammalian cell is a mammalian pancreatic β cell or a mammalian hepatocyte. In still another embodiment, the mammalian cell is a cancerous cell. In yet a further embodiment, the mammalian cell is in vitro. In still a further embodiment, the mammalian cell is located in a mammalian subject in need of increasing glycerol production and glycerol release from the mammalian cell and the method further comprises administering a therapeutically effective amount of the agent to the mammalian subject. In still another embodiment, the mammalian subject is afflicted by obesity, type II diabetes and/or metabolic syndrome X. In yet a further embodiment, the mammalian subject is afflicted by a cancer.

In a third aspect, the present disclosure provides an agent capable of increasing glycerol production and glycerol release from a mammalian cell. The agent is capable of increasing the biological activity of a mammalian glycerol-3-phosphate phosphatase (hG3PP) of in the mammalian cell. The agent can be, for example, succinic acid, a succinate salt and/or a nucleic acid expression system encoding the hG3PP. In an embodiment, the mammalian cell is a mammalian pancreatic β cell or a mammalian hepatocyte. In another embodiment, the mammalian cell is a cancerous cell. In yet another embodiment, the mammalian cell is in vitro. In still a further embodiment, the mammalian cell is located in a mammalian subject. In another embodiment, the mammalian subject is afflicted by obesity, type II diabetes and/or metabolic syndrome X. In still a further embodiment, the mammalian subject is afflicted by a cancer.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to 1G. Identification of phosphoglycolate phosphatase as a glycerol-3-phosphate phosphatase and its effect on glycerol release in rat islets and INS832/13 β cells. (A and B) Glycerol and FFA release (2 h) by isolated rat islets at 4, 10, 16 and 25 mM glucose with (▪) and without (●) 50 μM orlistat. Means±SEM of 4 independent experiments with triplicate observations. **P<0.01; ***P<0.001. (C) Kinetics of Gro3P hydrolysis by purified hPGP. Mean±SEM from 8 experiments. (D and E) RNAi knockdown of PGP/G3PP in INS832/13 cells reduces glucose induced glycerol release. (D) PGP/G3PP protein expression 48 h after transfection with 20 nM and 50 nM G3Pase siRNA or control siRNA or in not transfected cells (NT). (E) Glycerol release (2 h) with (right bar) and without RNAi knockdown (left and middle bars) at 2, 5, 10 and 20 mM glucose. Mean±SEM; n=4; **P<0.01 vs control (CTL) cells. (F and G) Overexpression of hG3Pase in INS832/13 cells enhances glucose induced glycerol release. (F) hPGP/G3PP protein expression after transfection with GFP and G3PP expression plasmids and in non-transfected cells. (G) Glycerol release with (left bars) and without (right and middle bars) overexpressed hG3PP at 2, 5, 10 and 20 mM glucose. Mean±SEM; n=4; **P<0.01; ***P<0.001 vs GFP control cells.

FIGS. 2A to 2H. Activity of G3PP controls glucose stimulated insulin secretion, glucotoxicity and glucolipotoxicity in β-cells. (A and B) Insulin secretion in INS832/13 cells at 2 and 10 mM glucose, after G3PP knockdown (A) or hG3PP overexpression (B). NT, not transfected; CTL, control. Mean±SEM of 3 experiments with triplicate observations. *P<0.05; **P<0.01 compared to corresponding controls. (C and D) Insulin secretion in isolated rat islets at 4 and 16 mM glucose, after G3PP knockdown (C) or hG3PP overexpression (D). Mean±SEM of 3 experiments with triplicate observations. *P<0.05; ** P<0.01 compared to corresponding controls. (E and F) Glucose-induced apoptosis (glucotoxicity) in INS832/13 cells, after G3PP knockdown for 24 h (E) or hG3PP overexpression for 72 h (F). Caspase activity was determined in cells exposed to 5 and 20 mM glucose. Mean±SEM of 3 experiments with triplicate observations. *P<0.05 compared to corresponding controls. (G) Glucose plus plamitate induced apoptosis (glucolipotoxicity) in INS832/13 cells, after G3PP RNAi-knockdown without or with rescue by hG3PP overexpression. Controls were set-up with control siRNA for knockdown and GFP for overexpression. Glucolipotoxicity was induced for 48 h by 20 mM glucose plus 0.3 mM palmitate and compared to 5 mM glucose value. Means±SEM of 3 experiments with triplicate observations. *p<0.05; **p<0.01 compared to corresponding controls; control siRNA, shG3PP and GFP. (H) Scheme illustrating the central role of G3PP in intermediary metabolism. Glycerol-3-phosphate formed from glucose metabolism or by the phosphorylation of lipolysis derived glycerol is at the crossroads of intermediary metabolism. G3PP, by controlling glycerol-3-phosphate, plays a central role in the regulation of intermediary and energy metabolism. Ac-CoA, acetyl-CoA; CE, cholesterol ester; Chl, free cholesterol; DAG, diacylglycerol; ETC, electron transport chain; FA-CoA, fatty acyl-CoA; FFA, free fatty acid; G3PP, glycerol-3-phosphate phosphatase; GK, glycerokinase; GL/FFA cycle, glycerolipid/free fatty acid cycle; LPA, lysophosphatidic acid; MAG, monoacylglycerol; OxPhos, oxidative phosphorylation; PA, phosphatidic acid; PL, phospholipids; Pyr, pyruvate; TCA cycle, tricarboxylic acid cycle; TG, triglyceride.

FIGS. 3A to 3I. Changes in G3PP expression modulate glucose, lipid and energy metabolism in β-cells. (A and B) Effect on fatty acid esterification at 2 and 10 mM glucose. (A) RNAi knockdown of G3PP and (B) overexpression of hG3PP in INS832/13 cells. DAG, diacylglycerol, TG, triglyceride, PL, phospholipids and LPA, lysophosphatidic acid. siRNA and GFP controls indicated. Mean±SEM; n=9; *P<0.05; **P<0.01; ***P<0.001. (C) Palmitate oxidation in INS832/13 cells at 2 and 10 mM glucose. NT, not transfected. Mean±SEM; n=6. (D) FFA release from INS832/13 cells at 2 and 10 mM glucose. Mean ±SEM; n=6; *P<0.05. (E) Glycerol release from rat islets at 4 and 16 mM glucose following RNAi knockdown of G3PP with lentiviral-shG3PP or hG3PP overexpression with adenoviral-hG3PP. NI, not infected. Mean±SEM; n=9; *P<0.05; **P<0.01. (F and G) Respiration and mitochondrial function in rat islets at 4 and 16 mM glucose following RNAi knockdown of G3PP (F) or hG3PP overexpression (G). Mean±SEM; n=9; *P<0.05; **P<0.01; ***P<0.001. (H and I) Western blot analysis of G3PP protein in rat islets after RNAi-knockdown (H) or overexpression of hG3PP (I).

FIGS. 4A to 4T. Effect of altered G3PP expression on liver metabolism in vitro and in vivo. (A-L) In vitro metabolic experiments with rat primary hepatocytes infected with lentivirus-shG3PP and control lentivirus-shGFP for G3PP knockdown, (A-E) or with adenovirus-hG3PP and control adenovirus-GFP for overexpression of hG3PP (F-J). (A and F) Gluconeogenesis from glycerol or pyruvate/lactate; (B and G) Palmitate oxidation at 5 and 25 mM glucose (5G and 25G); (C and H) Glycerol release; (D and I) Lactate production (intra cellular content); (E and J) Lactate release; (K and L) Fatty acid esterification using 1-14C-palmitate. 1,2(2,3)-Diacylglycerol (DAG) and triglyceride (TG) synthesis in hepatocytes with G3PP knockdown (K) or with hG3PP overexpression (L). Mean±SEM; n=6-8; *P<0.05; **P<0.01; ***P<0.001 compared to shGFP or GFP controls. (M-T) In vivo study of the effect of hG3PP overexpression. Rats were injected with adenovirus expressing hG3PP (n=6 shown as ▪) or GFP (n=5 shown as ●) and on day 7, glycerol load test was performed. Expression of hG3PP in liver was assessed and plasma glycerol and TG levels were measured prior to glycerol load. (M) hG3Pase mRNA and (N) hG3PP protein levels (representative Western blots from three separate rats). (O) Body weight and (P) net body weight gain in 7 days after adenoviral administration. (Q) Cumulative food intake. (R) Plasma glycerol and (S) triglyceride levels on day 7 after virus injection in 12 h fasted rats, prior to glycerol load. (T) Glycerol load test in rat expressing hG3PP or control GFP, to assess glycerol-derived glucose production. Blood was collected at indicated times following gavage of glycerol. Mean±SEM; *P<0.05; **P<0.01; ***P<0.001.

FIG. 5A to 5E. (A and B) Effect of panlipase inhibitor orlistat (▪) on glycerol and FFA release in INS832/13 cells at different glucose concentrations. (A) Release of glycerol and (B) FFA following 2 h incubation with and without 50 μM orlistat. Means±SEM of 3 experiments with triplicate observations; *P<0.05. (C) Reduction of PGP/G3PP in INS832/13 cells with three separate siRNAs is associated with lowered glycerol release. Upper panel: siRNA 1, 2 and 3 reduce the PGP/G3PP protein level as compared to the control siRNAs C1 and C2. NT, not transfected. Lower panel: Effect of various G3Pase siRNA on glycerol release. G3PP-siRNA1 and control siRNA-1 were used for rest of the study. Means±SEM of 3 experiments with triplicate observations; ***P<0.001 compared with C1 and C2. (D and E) Overexpression of hG3PP counters the effect of PGP/G3PP RNAi-knockdown on glycerol release and insulin secretion in INS832/13 cells. (D) Glycerol release at 2 and 10 mM glucose in cells transfected with G3PP-siRNA or control si-RNA, and with plasmid expressing GFP or hG3PP or empty vector. (E) Insulin secretion measured at 2 and 10 mM glucose in cells transfected with G3PP-siRNA or control si-RNA, and with plasmid expressing GFP or hG3PP. Means±SEM of 3 independent experiments with triplicate observations. *P<0.05; **P<0.01 ***P<0.001 compared with corresponding controls.

FIGS. 6A to 6F. Regulation of G3PP expression by nutritional status in mice and tissue distribution of G3PP in rats, related to FIG. 2. (A and B) G3PP expression in the fed and fasted states in various tissues. Male mice were fed normal chow diet and one group was starved overnight before sacrifice. Tissues were isolated and G3PP expression was measured. (A) G3PP mRNA levels normalized to corresponding tissue cyclophilin mRNA. (B) G3PP protein expression in different tissues assessed by Western blots and densitometry. G3PP protein levels were normalized to corresponding tissue levels of β-actin or α-tubulin and expressed as fold change in expression. Means±SEM; n=6; *P<0.05. (C and D) Effect of high-fat diet on G3PP expression in various tissues. Male mice were fed normal diet (ND) or high fat diet (HFD, 60% calories from fat) for 8 weeks and then sacrificed, and tissues were collected for assessing G3PP expression. (C) G3PP mRNA levels. (D) G3PP protein levels. Means±SEM; n=5-10; *P<0.05; **P<0.01. Sk. muscle, skeletal muscle; BAT, brown adipose tissue; VAT, visceral adipose tissue; SAT, subcutaneous adipose tissue. (E) Expression of G3PP mRNA in normal Wistar rat tissues and in the rat β-cell line IN832/13. Means±SEM; n=4. (F) Expression of G3PP protein in different rat tissues. Representative blot of 3 experiments.

FIGS. 7A to 7D. Altered G3PP expression in INS832/13 cells affects lipid and energy metabolism, related to FIG. 3. (A and B) Fatty acid esterification to 1,3-DAG, lysophosphatidylinositol (LPI) and lysophosphatidylcholine (LPC). (A) RNAi knockdown of G3PP and (B) overexpression of hG3PP. Fatty acid esterification was measured using [1-¹⁴C]-palmitate at 2 and 10 mM glucose. (C and D) Oxygen consumption, ATP production and H⁺ leak. Respiratory measurements in transfected cells were made at 2 and 10 mM glucose, using Seahorse XF-analyzer and ATP production and H+leak were calculated. (C) RNAi-knockdown of G3PP and (D) hG3PP overexpression. Means±SEM; *P<0.05; **P<0.01; ***P<0.001 versus GFP or control siRNA groups; n=12.

FIGS. 8A to 8G. Effect of altered G3PP expression in rat primary hepatocytes and on lipid metabolism, and related to FIG. 4. (A-D) Rat primary hepatocytes were infected with lentivirus-shG3PP and control lentivirus-shGFP for G3PP knockdown or with adenovirus-hG3PP and control adenovirus-GFP for overexpression of hG3PP. (A and B) Western blot analysis of G3PP expression after RNAi-knockdown (A) and hG3PP overexpression (B). (C and D) Fatty acid esterification using 1-¹⁴C-palmitate. (C) Cholesterol ester (CE) and phospholipid (PL) synthesis with G3PP knockdown. (D) Cholesterol ester (CE) and phospholipid (PL) synthesis with hG3PP overexpression. Means ±SEM; n=6; *P<0.05 compared to shGFP or GFP. (E and F) Low density lipoprotein (LDL) and high density lipoprotein (HDL) levels in the plasma of rats 6 days after injection with Adv-G3PP (n=6) or Adv-GFP control. Means±SEM; n=5; *P<0.05 vs Adv-GFP. (G) Western blot analysis of G3PP expression in different tissues, with α-tubulin as loading control. Representative blots of 3 hG3Pase- and 3 GFP-adenovirus-injected rats. VAT, visceral adipose tissue; BAT, brown adipose tissue; RBC, red blood cells.

FIGS. 9A to 9C. Effect of various organic acids on G3PP activity. The effect of various organic acids, at two concentrations (2 mM and 10 mM), was determined in cellular extracts. Effect of hydroxybutyric acid (A and B), succinate (B and C), citrate (B), lactate (B), malate (B and C), oxaloacetate (B), sodium glycolate (B), fumarate (B) and malonic acid (C) on G3PP activity. Results are shown as nmol glycerol/min/mg extract in function of experimental conditions. Blank =Enzyme reaction measured with G3PP extracts without adding the substrate G3P. GFP=control-GFP overexpressed extract with the substrate G3P. Extract=G3PP-expressing cell extracts with the G3P substrate.

FIGS. 10A to 10D. Effect of hG3PP overexpression on cancer cell viability and proliferation. A549 lung cancer cells (3×10⁵ cells per well), HeLa cervical cancer cells (1.5×10⁵ cells per well) and PC3 prostate cancer cells (3×10⁵ cells per well) were seeded in 6-well plates at precisely the same density in control (GFP) and test (G3PP) wells and the next day the cells were infected with adenoviral vectors expressing either hG3PP or Green Fluorescent Protein (GFP) as control. After 72 h following infection and overexpression all the cells in each well (floating plus attached) were harvested and counted using Trypan Blue stain and hematocytometer. Cell viability and total number of cells for each cancer cell line were calculated. Results are shown as number of cells. (A) A549 lung cancer cells, (B) HeLa cervical cancer cells and also the (C) PC3 prostate cancer cells. There were many floating dead cells when the cells were overexpressing hG3PP, as compared to GFP overexpressing cells. An example of this was shown with PC3 cells (D). *P<0.05 vs corresponding GFP expressing control cells.

FIGS. 11A to D. Effect of hG3PP overexpression on pancreatic cancer cells. Two different cell lines (MiaPaCa2 and PANC-1) were infected to express hG3PP (adPGP or black bars) or the control green fluorescent protein (adGFP or gray bars). Apoptosis (A), DNA content (B), cell proliferation (C) and lactate production (D) were measured and compared.

### DETAILED DESCRIPTION

Glycerol release from mammalian cells is thought to occur exclusively from the lipolytic segment of the GL/FA cycle and glycerol production is considered to reflect lipolysis flux. It was previously proposed that at high glucose concentrations the release of glycerol by β-cells, which do not express glycerokinase that transforms glycerol to Gro3P (Prentki and Madiraju, 2012), is a mechanism of “glucolipodetoxification” and that this process is dependent on the lipolysis segment of GL/FA cycle (Prentki and Madiraju, 2008, 2012). The fate of Gro3P in mammalian cells is thought to be its conversion to either dihydroxyacetone phosphate (DHAP) or lysophosphatidate, the first intermediate of the lipogenic arm of the cycle. However, many microbes and plants harbor a G3PP. In animals, it has been reported that preparations of fish liver (Ditlecadet and Driedzic, 2013), rat heart (De Groot et al., 1994) and rat brain (Nguyen et al., 2007), can generate glycerol and inorganic phosphate from Gro3P. However, the molecular identity of the mammalian enzyme(s) responsible for this catalytic activity, and their physiological significance, are unknown. In the present disclosure, it is described that a previously known phosphoglycolate phosphatase (PGP) with an uncertain function in mammalian cells acts as a specific mammalian Gro3P phosphatase (hG3PP) and plays a pivotal role in the regulation of glucose and lipid metabolism and signaling as well as in the response to metabolic stress.

### Screening Applications

As shown herein, the activity of hG3PP in mammalian cells, and especially in pancreatic β cells and in hepatocytes as well as in cancer cells, is tightly linked to glucose and lipid metabolism as well as response to metabolic stress(es). The experimental data presented herewith elegantly show that activation of hG3PP favors glycerol release, limits glucose synthesis and modulates lipid signaling. In vivo, the activation of hG3PP is associated with a decrease in body weight, a decrease in (cumulative) food intake, an increase in plasma glycerol, a decrease in plasma triglycerides and a decrease in gluconeogenesis. As such, potential therapeutic agents capable of increasing the expression and/or the biological activity of the hG3PP are believed to be able to mediate similar biological effects in vitro and in vivo. Thus, the present disclosure relates to a method of characterizing an agent's ability for increasing glycerol formation from Gro3P in and/or release from a mammalian cell. The method can be used to identify agents capable of limiting or preventing gluconeogenesis in a mammal or a mammalian cell. Those agents can be particularly useful for the treatment, alleviation of symptoms or prevention of diabetes (such as type II diabetes) or any other condition associated with heightened gluconeogenesis caused (at least in part) by the formation of increased levels of Gro3P from glycerol (such as obesity and metabolic syndrome X). Those agents can also be useful for the treatment, alleviation of symptoms or prevention of a cancer.

The assay described herein, in an embodiment, comprises the use of a human glycerol-3-phosphate phosphatase (also referred to as hG3PP). As used in the context of the present disclosure, hG3PP refers to a polypeptide having phosphatase activity. In some embodiments, hG3PP refers to a polypeptide capable, under the appropriate conditions, of dephosphorylating glycerol-3-phosphate. The hG3PP can be provided as a mammalian cellular extract (an extract of a cell or tissue, known to express hG3PP). In some embodiments, the cellular extract can be obtained from a pancreas, a pancreatic islet, a pancreatic β cell or a pancreatic cell line (such as, for example, INS 832/13, MIN6, INS1, RINm5F or HIT, etc.). In another embodiment, the cellular extract is obtained from a liver, an hepatocyte or an hepatocyte cell line such as, for example, rat hepatoma HIIE cells and human HepG2). In still another embodiment, the cellular extract is obtained from a cancerous cell or a cell line derived therefrom (such as, for example, a carcinoma cell, including, but not limited to, a breast carcinoma cell (A549 or Hela cells for example), a prostate carcinoma cell (PC3 cells for example) or a pancreatic carcinoma cell (MiaPaCa or PANC1 cells for example)). In an alternative embodiment, the hG3PP can be obtained in a substantially purified form or an isolated form. As used in the context of the present disclosure, hG3PP provided in “a substantially purified form” or “isolated form” refers to a hG3PP polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. Ordinarily, however, isolated hG3PP polypeptide will be prepared by at least one purification step. The “substantially purified” or “isolated” hG3PP can be obtained from a source endogenously expressing the hG3PP polypeptide (such as the mammalian pancreas or the liver) or from the recombinant expression in a transgenic host cell (genetically modified to expression the hG3PP polypeptide).

As used herein, hG3PP is a biological proteinaceous entity that can be derived from the hG3PP polypeptide itself or its corresponding nucleotide. The hG3PP polypeptide may be obtained from humans (such as, for example, as indicated in GenBank Accessions Nos. NP_001035830, NP_077023, XP_001130630 and XP_001130859). When the hG3PP polypeptide is obtained from a transgenic host, the nucleic acid molecule encoding the hG3PP polypeptide can be codon-optimized for facilitating its expression or recovery from the transgenic host.

In the context of the present disclosure, hG3PP can be the full-length hG3PP polypeptide or a biologically active fragment of the hG3PP polypeptide that retains its characteristic phosphatase activity. “Fragments” or “biologically active portions” of the hG3PP polypeptide include polypeptide fragments comprising amino acid sequence sufficiently identical to or derived from the amino acid sequence of the hG3PP polypeptide and exhibiting at least one activity of the hG3PP polypeptide (such as hG3PP-specific phosphatase activity), but which include fewer amino acids than the full-length hG3PP polypeptide (at least one amino acid is missing). The hG3PP can be result, for example, from truncation of the native hG3PP from the amino terminus, from the carboxy terminus or both. The hG3PP can also result, for example, from an internal deletion of amino acids (coupled or not with a N- and/or C-truncation). Typically, biologically active portions comprise a domain or motif with at least one activity (such as phosphatase activity) of the hG3PP polypeptide. A biologically active portion of the hG3PP polypeptide can be a polypeptide that is, for example, 10, 25, 50, 100, 150, 200, 250, 300 or more amino acids in length. Such biologically active portions can be prepared by recombinant techniques and evaluated for one or more of the functional activities (such as phosphatase activity) of a native hG3PP polypeptide.

Still in the context of the present disclosure, hG3PP can be a variant of the hG3PP polypeptide. By “variants” is intended polypeptides having an amino acid sequence that is at least about 45%, 55%, 65%, preferably about 75%, 85%, 95%, or 98% identical to the hG3PP polypeptide. The degree of identity can be determined over the entire length of the hG3PP polypeptide. Such variants generally retain the functional activity (e.g., phosphatase) of the hG3PP polypeptide. Variants do include conservative amino acid modifications. A “conservative amino acid substitution” is one in which at least one amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Variants also include polypeptides that differ in amino acid sequence due to natural allelic variation or intentional mutagenesis.

A biologically active fragment of the hG3PP polypeptide or a variant of the hG3PP polypeptide does not need to have the level of phosphatase activity as the full-length native hG3PP polypeptide, but most retain at least some phosphatase activity (at least 50%, at least 60%, at least 70%, at least 80%, at least 90%, or even at least 95% of the phosphatase activity of the full-length native hG3PP polypeptide). A biologically active fragment of the hG3PP polypeptide or a variant of the hG3PP polypeptide does not need to have the spectrum of phosphatase activity as the full-length native hG3PP polypeptide, but must retain catalytic activity against at least one cognate substrate of the full-length native hG3PP polypeptide (glycerol-3-phosphate for example). Preferably, the biologically active hG3PP fragment or variant should be able to have some enzymatic activity towards glycerol-3-phosphate.

The assays described herein can also rely on a hG3PP chimeric or fusion protein. As used herein, the “chimeric protein” or “fusion protein” comprises the hG3PP polypeptide (including the native, the fragments or the variants thereof) operably linked to a non-hG3PP polypeptide. A “non-hG3PP polypeptide” is intended to refer to a polypeptide having an amino acid sequence corresponding to a protein that is not substantially identical to the hG3PP polypeptide, e.g., a protein that is different from the hG3PP polypeptide and which is derived from the same or a different organism. The non-hG3PP polypeptide can be fused to the N-terminus or C-terminus of the hG3PP polypeptide, fragment or variant.

The first step of the method also includes providing a substrate that is recognized and cleaved by the hG3PP polypeptide, fragment or variant. In the context of the present disclosure, a substrate that can be “recognized and cleaved” by the hG3PP is capable of being physically associated with the hG3PP polypeptide, fragment or variant and of being cleaved by the hG3PP polypeptide. As indicated above, the hG3PP polypeptide (as well as its corresponding fragments and variants) exhibits phosphatase activity and as such is capable of cleaving the bond between a first moiety (glycerol for example) and a phosphate group (a single phosphate group for example). In some embodiments, the hG3PP polypeptide (as well as its corresponding fragments and variants) is capable of cleaving a bond between glycerol and phosphate, such as, for example, the bond between glycerol and phosphate which is present in glycerol-3-phosphate. The person skilled in the art will recognized that other commercially available substrates for the hG3PP polypeptide can be used, for example, p-nitrophenylphosphate or 6,8-difluoro-4-methylumbelliferyl phosphate (DiFMUP).

The substrate that is cleaved by the hG3PP polypeptide, fragment or variant contains at least one “detectable moiety”. In the context of the present disclosure, a “detectable moiety” is a moiety present in the substrate that can be detected or can no longer be detected once the substrate has been cleaved by the hG3PP polypeptide, fragment or variant. The detectable moiety can be detected because its physico-chemical properties are modified upon cleavage making it detectable in a cleaved form and undetectable in an uncleaved form (and vice versa). In some embodiments, the detectable moiety contains a label that can be detected either because the substrate is in an uncleaved or has been cleaved. The label can be, for example, a colorimetric label, a radioactive label or a fluorescent label. In some embodiments, the label is a fluorescent label. In other embodiments, the detectable moiety is one of the cleavage product obtained by the enzymatic action of the hG3PP on the substrate. The cleavage product can be detected directly (for example by attaching a label thereto) or indirectly (for example by measuring its presence via reaction with chemical reagents or via the binding of an antibody or via a further enzymatic activity). For example, the moiety can be detected enzymatically (for example using a radiometric method) and/or using antibody-based or lectin-based assays (such as, for example, ELISA). In some embodiments, when the substrate is glycerol-3-phosphate, the detectable moiety can be glycerol. In such embodiments, glycerol can be detected by a radiometric method or an enzymatic method (by using glycerokinase and a Gro3P oxidase for example). In further embodiments, glycerol can be detected using a radiometric method involving the use of [Γ-³²P]ATP and glycerokinase. In other embodiments, when the substrate is glycerol-3-phosphate, the detectable moiety can be inorganic phosphate.

Once the hG3PP and its substrate have been provided, they are combined with a test agent. In some advantageous embodiments, the test agent can be combined with the hG3PP prior to the addition of the substrate. Even though such combination can be made in vivo or ex vivo, it is preferred that the combination occurs in vitro, and in some embodiments, outside the context of a cell (for example, when the hG3PP is provided as a cellular extract or in a purified form). The combination between the test agent, the hG3PP and the hG3PP substrate must be made under conditions allowing the cleavage of the substrate. In some embodiments, it may be advantageous to provide the substrate at or near a saturating concentration (for example 10 mM when glycerol-3-phosphate is used as a substrate for the full-length native hG3PP polypeptide). Using a substrate at or near a saturating concentration allows for the identification of activators capable of enhancing the enzymatic turnover, i.e., Vₘₐₓ. In an embodiment, “near” saturating concentration is the concentration of Gro3P at which hG3PP shows 80 to 85% of Vₘₐₓ activity, while saturating concentration is where G3PP shows 100% Vₘₐₓ activity. Alternatively, in the context of the present disclosure, “near” saturating can be from 0.5-8 mM or from 2-8 mM, while saturating concentration is above 8 mM of Gro3P. In other embodiments, the conditions can include incubating the test agent, the hG3PP and the hG3PP at a temperature of about 30° C. In still another embodiment, the conditions can include incubating the test agent, the hG3PP and the hG3PP in the presence of Mg²⁺ ions, for example at a concentration between about 2 and 5 mM. In still another embodiment, the conditions can include incubating the test agent, the hG3PP and the hG3PP at a temperature of about 30° C. and in the presence of Mg²⁺ ions, for example at a concentration between about 2 and 5 mM.

Once the test agent has been combined with the hG3PP polypeptide, fragment or variant as well as the substrate, a test amount of the detectable moiety is determined. As indicated above, this determination or measurement can be made because the substrate has at least one a detectable moiety. The determination of the test amount of the detectable moiety can be made by measuring the amount of a label associated with the detectable moiety, the amount of a label associated with the uncleaved substrate, the amount of an enzymatic reaction product associated with the at least one detectable moiety, the amount of binding of a specific analyte to the detectable moiety, or the amount of reaction of a specific analyte with the detectable moiety. This determination may be made directly in the reaction vessel in which the test agent is combined with the hG3PP polypeptide, fragment or variant and the substrate or on a sample of such reaction vessel. In an embodiment, at least one detectable moiety is glycerol and the amount of glycerol is determined enzymatically using a radiometric method. In another embodiment, the detectable moiety is inorganic phosphate.

The determination/measuring step can rely on the addition of a quantifier (or a label) specific to the detectable moiety. The quantifier can specifically bind to the detectable moiety. In those instances, the amount of the quantifier that specifically bound (or that did not bind) to the detectable moiety will be determined to provide a measurement of the test amount. In an embodiment, the signal of the quantifier can be provided by a label that is either directly or indirectly linked to a quantifier. The label can be, for example, colorimetric, radioactive or fluorescent.

Once the test amount has been determined, it is compared to a first control amount to determine the presence or absence of activation, by the test agent, of the biological activity of the hG3PP polypeptide, fragment or variant. In an embodiment, the first control amount is derived or obtained from combining hG3PP and the substrate in the absence of the test agent. This first control amount can be used to establish a base level of the biological activity of the hG3PP polypeptide, fragment or variant and allow the characterization of the test agent's potential for increasing the biological activity or activating the hG3PP polypeptide, fragment or variant. The first control amount may be the amount of the detectable moiety when the hG3PP polypeptide, fragment or variant is combined with the substrate (in the presence or absence of a control agent lacking the ability to modulate the hG3PP polypeptide, fragment or variant's biological activity). The first control amount may be a pre-determined value (or a set of predetermined values or range) derived from the amount of the detectable moiety when the hG3PP polypeptide, fragment or variant is combined with the substrate (in the presence or absence of a control agent lacking the ability to modulate the hG3PP polypeptide, fragment or variant's biological activity). In some embodiments, the first control amount can be measured prior to combining of the test agent with the hG3PP polypeptide, fragment or variant and the substrate or in two replicates of the same reaction vessel where one of the replicates does not comprise the test agent. Optionally, the method can comprise a step of providing such first control amount.

Once the comparison between the test amount and the control amount is made, then it is possible to characterize the test agent's ability to increase the conversion of Gro3P to glycerol in the mammalian cell and/or increase glycerol release from the mammalian cell. This characterization is possible because, as shown herein, an agent that increases or activates the biological activity of the hG3PP polypeptide, fragment or variant favors glycerol synthesis and release and ultimately impedes gluconeogenesis.

In some embodiments, the method also comprises comparing a second control amount to the first control amount to ascertain the validity of the results that are being obtained and make sure that the assay is capable of detecting increases in the biological activity of the hG3PP polypeptide, fragment or variant. This second control amount is obtained or derived from combining a control agent known to increase the biological activity of the hG3PP polypeptide, fragment or variant with the hG3PP polypeptide, fragment or variant and the substrate and determining the second control amount obtained for the detectable moiety. Such control agent can be, for example, succinate or a succinate salt. As shown herein, succinate increases the biological activity of the hG3PP polypeptide, fragment or variant. Once the second control amount of the detectable moiety has been obtained, it is compared with the first control amount of the detectable moiety. If the second control amount of the detectable moiety is determined to be higher than the first control amount of the detectable moiety, then the screening assay is characterized as being adequate for determining increases in the biological activity of the hG3PP polypeptide, fragment or variant. As such, any previous characterization of the test agent is considered to be valid. On the other hand, if the second control amount of the detectable moiety is determined to be the same or lower than the first control amount of the detectable moiety, then the screening assay is characterized as being flawed for determining increases in the biological activity of the hG3PP polypeptide, fragment or variant. As such, any previous characterization of the test agent is not considered to be valid. In some embodiments, the methods described herein can also including providing such second control amount.

In some embodiments, the methods and assays described herein can be used to characterize the ability of a test agent to increase the conversion of Gro3P to glycerol and glycerol release from mammalian pancreatic β cells. In such embodiment, test agents characterized as being useful for increasing conversion of Gro3P to glycerol and glycerol release will also be considered useful for controlling glucose-stimulated insulin secretion, for decreasing glucotoxicity, for decreasing glucolipotoxicity, for decreasing diacylglycerol synthesis, for decreasing triglyceride synthesis, for decreasing phospholipid synthesis, for decreasing lysophosphatidic acid synthesis, for decreasing oxygen consumption, for decreasing ATP production and/or for increasing free fatty acid release in the mammalian pancreatic β cell. In alternative embodiments, the methods and screening assays described herein can also include a step of determining the effect of the test agent, in a mammalian pancreatic β cell, on the amount glucose-stimulated insulin secretion, the level glucotoxicity, the level of glucolipotoxicity, the amount of diacylglycerol synthesis, the amount of triglyceride synthesis, the amount of phospholipid synthesis, the amount of lysophosphatidic acid synthesis, the amount of oxygen consumption, the amount of ATP production and/or the level free fatty acid release. If it is determined that the test agent, in the mammalian pancreatic β cell, causes a decrease in glucose-stimulated insulin secretion, a decrease in glucotoxicity, a decrease in glucolipotoxicity, a decrease in diacylglycerol synthesis, a decrease in triglyceride synthesis, a decrease in phospholipid synthesis, a decrease in lysophosphatidic acid synthesis, a decrease in oxygen consumption, a decrease in ATP production and/or an increase in free fatty acid release, then it is confirmed that the test agent is capable of increasing Gro3P conversion to glycerol and glycerol release in the mammalian pancreatic β cell. On the other hand, if it is determined that the test agent, in the mammalian pancreatic β cell, causes an increase or no change in glucose-stimulated insulin secretion, an increase in glucotoxicity, an increase in glucolipotoxicity, an increase in diacylglycerol synthesis, an increase in triglyceride synthesis, an increase in phospholipid synthesis, an increase in lysophosphatidic acid synthesis, an increase in oxygen consumption, an increase in ATP production and/or a decrease in free fatty acid release, then it is confirmed that the test agent is not capable of increasing Gro3P conversion to glycerol and glycerol release in the mammalian pancreatic β cell.

In some embodiments, the methods and assays described herein can be used to characterize the ability of a test agent to increase Gro3P conversion to glycerol and glycerol release from a mammalian hepatocyte. In such embodiment, test agents characterized as being useful for increasing Gro3P conversion to glycerol and glycerol release will also be considered useful for decreasing gluconeogenesis, for decreasing lactate synthesis, for decreasing lactate release, for decreasing diacylglycerol synthesis, for decreasing triglyceride synthesis and/or for increasing free fatty acid oxidation in the mammalian hepatocyte. In alternative embodiments, the methods and screening assays described herein can also include a step of determining the effect of the test agent, in a mammalian hepatocyte, on the amount of glucose synthesis, the amount of lactate synthesis, on the amount of lactate release, on the amount of diacylglycerol synthesis, on the amount of triglyceride synthesis and/or on the level free fatty acid oxidation. If it is determined that the test agent, in the mammalian hepatocyte, causes a decrease in gluconeogenesis, a decrease in lactate synthesis, a decrease in lactate release, a decrease in diacylglycerol synthesis, a decrease in triglyceride synthesis and/or an increase in free fatty acid oxidation, then it is confirmed that the test agent is capable of increasing Gro3P conversion to glycerol and glycerol release in the mammalian hepatocyte. On the other hand, if it is determined that the test agent, in the mammalian hepatocyte, causes an increase or no change in gluconeogenesis, an increase or no change in lactate synthesis, an increase or no change in lactate release, an increase or no change in diacylglycerol synthesis, an increase or no change in triglyceride synthesis and/or a decrease in free fatty acid oxidation, then it is confirmed that the test agent is not capable of increasing Gro3P conversion to glycerol and glycerol release in the mammalian hepatocyte.

In some embodiments, the methods and assays described herein can be used to characterize the ability of a test agent to increase Gro3P conversion to glycerol and glycerol release from a mammalian cancer cell. In such embodiment, test agents characterized as being useful for increasing Gro3P conversion to glycerol and glycerol release will also be considered useful for decreasing proliferation and/or survival of cancer cells, which are dependent on glycolysis for energy and high levels of Gro3P for lipid synthesis, needed for cell growth and multiplication.

As shown herein, the level of biological activity of the hG3PP polypeptide, fragment or variant is tightly linked to Gro3P conversion to glycerol and glycerol release from mammalian cells. The experimental data presented herewith elegantly show that upregulation the biological activity of the hG3PP polypeptide, fragment or variant favors Gro3P conversion to glycerol and glycerol release and ultimately impedes gluconeogenesis. As such, the present disclosure relates to a method of characterizing an agent's ability for increasing Gro3P conversion to glycerol and glycerol release in a mammalian cell (which can optionally be present in a mammalian subject). Those agents can be particularly useful for the treatment, alleviation of symptoms or prevention of diabetes (such as type II diabetes) or any other condition associated with a low level of glycerol synthesis/release (such as obesity and metabolic syndrome X). Those agents can be particularly useful for the treatment, alleviation of symptoms or prevention of cancer.

### Therapeutic Applications

It has been shown herein that an increase in the biological activity of hG3PP causes a decrease in Gro3P, an associated increase in Gro3P conversion to glycerol, an increase in glycerol release from the mammalian cell which will ultimately impede or limit gluconeogenesis. These biological effects can be useful in the prevention, treatment or alleviation of symptoms of conditions associated with low level of Gro3P conversion to glycerol and low glycerol release and/or high gluconeogenesis. As such, therapeutic agents capable of increasing the expression and/or the catalytic activity of hG3PP can be successfully used in the prevention, treatment or alleviation of symptoms of conditions associated with a low level of Gro3P conversion to glycerol and low glycerol release and/or high gluconeogenesis. Optionally, these therapeutic agents can be identified by the screening method proposed herewith.

As used in the context of the present disclosure, the expression “prevention, treatment or alleviation of symptoms” collectively refer to the ability of a therapeutic agent to limit the development, progression and/or symptomology of conditions associated with a low level of Gro3P conversion to glycerol and low glycerol release and/or high gluconeogenesis. Broadly, the prevention, treatment and/or alleviation of symptoms can encompass the reduction of gluconeogenesis.

One of the conditions associated with low Gro3P conversion to glycerol and low glycerol release and/or high gluconeogenesis is diabetes. Diabetes can be divided into two broad type of diseases: type I and type II diabetes. Type II diabetes (also referred to as non-insulin-dependent diabetes mellitus (NIDDM), adult-onset diabetes or diabetes mellitus type II) is a disorder that is characterized by high blood glucose in the context of insulin resistance and relative insulin deficiency. Unlike type I diabetes, there is very little tendency toward ketoacidosis in type II diabetes.

Another condition associated with associated with low glycerol synthesis, low glycerol release and/or high gluconeogenesis is metabolic syndrome X. Metabolic Syndrome X is generally used to define a constellation of abnormalities that is associated with increased risk for the development of type II diabetes and atherosclerotic vascular disease. Metabolic Syndrome X is can also be referred to, in the art, as “metabolic syndrome,” “insulin resistance syndrome,” and “syndrome X”. Risk factors include, but are not limited to, central obesity, sedentary lifestyle, aging, diabetes mellitus, coronary heart disease and lipodystrophy. Related conditions and symptoms include, but are not limited to, fasting hyperglycemia (diabetes mellitus type II or impaired fasting glucose, impaired glucose tolerance, or insulin resistance), high blood pressure; central obesity (also known as visceral, male-pattern or apple-shaped adiposity), overweight with fat deposits mainly around the waist; decreased HDL cholesterol; elevated triglycerides. Associated diseases can also include hyperuricemia, fatty liver (especially in concurrent obesity) progressing to non-alcoholic fatty liver disease, polycystic ovarian syndrome (in women), and acanthosis nigricans.

A further condition associated with associated with a low level of Gro3P conversion to glycerol and low glycerol release and/or high gluconeogenesis is obesity. Overweight and obesity are defined as abnormal or excessive fat accumulation that presents a risk to health. A crude population measure of obesity is the body mass index (BMI), a person's weight (in kilograms) divided by the square of his or her height (in meters). A person with a BMI of 30 or more is generally considered obese. A person with a BMI equal to or more than 25 is considered overweight. Overweight and obesity are major risk factors for a number of chronic diseases, including diabetes, cardiovascular diseases and cancer.

A further condition associated with associated with a low level of Gro3P conversion to glycerol and low glycerol release and/or high glycolysis is cancer. Cancer, also known medically as a malignant neoplasm, is a term for a large group of different diseases, all involving unregulated cell growth. In cancer, cells divide and grow uncontrollably, forming malignant tumors, and invade nearby parts of the body. The cancer may also spread to more distant parts of the body through the lymphatic system or bloodstream, a process called metastasis. In the context of the present disclosure, the term cancer encompasses carcinoma, sarcoma, lymphoma, leukemia: germ cell tumor and blastoma. The primary lesions can be identified with cellular markers which include but are not limited to, ALK, α-fetoprotein (AFP), β2-microglobulin (B2M), β-human chorionic gonadotropin (3-hCG), BCR-ABL fusion gene, BRAF mutation V600E, CA15-3/CA27.29, CA19-9, CA125, CEA, CD20, chromogranin A (CgA), cytokeratin fragments 21-1, EGFR mutations, Estrogen receptor/progesterone receptor, fibrin/fibrinogen, HE4, HER2/neu, KIT, KRAS mutations, nuclear matrix protein 22, PSA, thyroglobulin, uPA and PAI-1, Ova1, 70-gene signature (Mammaprint). Symptoms associated with cancer disorder include, but are not limited to: local symptoms which are associated with the site of the primary cancer (such as lumps or swelling (tumor), hemorrhage, ulceration and pain), metastatic symptoms which are associated to the spread of cancer to other locations in the body (such as enlarged lymph nodes, hepatomegaly, splenomegaly, pain, fracture of affected bones, and neurological symptoms), and systemic symptoms (such as weight loss, fatigue, excessive sweating, anemia and paraneoplastic phenomena). Cancer cells are very much dependent on high levels of glycolysis for energy purposes and also on the formation of glycolysis-dependent formation of Gro3P, needed for the synthesis of various lipid building blocks, required for membrane formation and cell growth and proliferation. Thus without active glycolysis and elevated supply of Gro3P, cancer cells survival and proliferation is limited. Therefore, accelerated hydrolysis of Gro3P in cancer cells to form glycerol, leads to reduced glycolysis-dependent energy production and lipid synthesis and thus curtailed cancer cell survival and proliferation. In embodiments, the cancer is a carcinoma, for example, from the breast, the prostate or the pancreas.

Broadly, the present disclosure provides an agent capable of increasing the biological activity of the hG3PP polypeptide (either directly or indirectly by increasing its catalytic activity or by increasing its level of expression) for decreasing the amount of Gro3P, increasing Gro3P conversion to glycerol and increasing glycerol release from a mammalian cell. The agent is contacted at an effective amount and under the appropriate conditions to increase the biological activity of the hG3PP polypeptide. The agent can be used to increase the biological activity of the hG3PP polypeptide of any cell or tissue expressing the hG3PP polypeptide. In some embodiments, the agent is capable of causing an increase the biological activity of the hG3PP polypeptide in a pancreatic β cell (or in the pancreas), an hepatocyte (or in a liver) or in a cancer cell (such as a carcinoma cell). The agent can be a “therapeutic” agent when used for mediating therapeutic benefits to a mammalian host. The agent can be a “screening” agent when used in screening assays. The agent can be a “reagent” when used in vitro for research purposes.

The agent can be used in vitro to cause an increase of the biological activity of the hG3PP polypeptide in a cellular extract (such as, for example, an extract from a pancreatic β cell or an hepatocyte), a cell (primary cell, immortalized cell, from a cell line) or a tissue-like structure comprise the cell (a pancreatic islet for example). In some instances, the agent can be used to increase the biological activity of the hG3PP provided in a substantially purified form. When used in vitro, the agent can be used to provide the second control amount described above that can be used in screening applications or can be used as a reagent.

The agent can be used in vivo in a mammalian subject in need of increasing Gro3P conversion to glycerol and glycerol release. For example, the mammalian subject can be in need of decreasing gluconeogenesis and glycolysis. In still another example, the mammalian subject can be afflicted by or suspected of being afflicted by type II diabetes, metabolic syndrome X or obesity. In yet another example, the mammalian subject can be afflicted by or suspect of being afflicted by a cancer. As such, a “therapeutically effective amount” of the therapeutic agent is intended to be administered to the mammalian subject. As used herein, the expression “therapeutically effective amount” is a dosage which is sufficient to increase the biological activity of the hG3PP polypeptide and achieve at least one therapeutic effect. In the context of the present disclosure, the therapeutic effects includes, but are not limited to glycemic control, reduction of fat mass, reducing hepatic steatosis, improving pancreatic beta cell function and survival, improvement in blood pressure parameters, reduction in cancerous tumors and/or metastasis (number and/or size), reduction in metastatic potential, etc. Generally, a therapeutically effective amount may vary with the mammalian subject's age, condition, and sex, as well as the extent of the condition in the subject and can be determined by a person skilled in the art. The dosage may be adjusted by the individual physician or veterinarian in the event of any complication. In embodiments wherein the agent acts to enhance the catalytic activity of hG3PP, the therapeutically effective amount typically will vary from about 0.01 mg/kg to about 500 mg/kg, typically from about 0.1 mg/kg to about 200 mg/kg, and often from about 0.2 mg/kg to about 20 mg/kg, in one or more dose administrations daily, for one or several days (depending of course of the mode of administration and the factors discussed herein).

The agents described herein can be incorporated into pharmaceutical compositions. Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Pharmaceutical compositions are typically formulated to be compatible with their intended route of administration. Examples of routes of administration include parenteral, e.g., intravenous, intradermal, subcutaneous, oral, transdermal, topical, transmucosal, and rectal administration.

In one embodiment, the agent can be an organic acid or a salt of an inorganic acid. Exemplary organic acids that can be used as an agent to increase the biological activity of the hG3PP polypeptide include, but are not limited to, β-hydroxybutyratic acid, succinic acid, citric acid, lactic acid, malic acid, oxaloacetic acid, glycolic acid, fumaric acid, malonic, malic acid as well as combinations of such organic acids. In some embodiments, the organic acids that can be used as an agent to increase the biological activity of the hG3PP polypeptide include, but are not limited to, β-hydroxybutyratic acid, succinic acid, citric acid, lactic acid, malic acid, oxaloacetic acid, glycolic acid, fumaric acid, malonic, malic as well as combinations of such organic acids. In some additional embodiment, the organic acids that can be used as an agent to increase the biological activity of the hG3PP polypeptide include, but are not limited to, β-hydroxybutyric acid, succinic acid as well as combinations of such organic acids. In yet a further embodiment, the organic acids that can be used as an agent to increase the biological activity of the hG3PP polypeptide is succinate.

The organic acid that can be used as the agent can be provided in the form of an acceptable salt. As such, the organic acid salts that can be used as an agent include, but are not limited to β-hydroxybutyrate, succinate, citrate, lactate, malate, oxaloacetate, glycolate, fumarate, malonate as well as combinations of these organic acid salts. In another embodiment, the organic acid salts that can be used as an agent include, but are not limited to 3-hydroxybutyrate, succinate, citrate, lactate, malate, oxaloacetate, glycolate, fumarate as well as combinations of these organic acid salts. In additional embodiments, the organic acid salts that can be used as an agent include, but are not limited to β-hydroxybutyrate and succinate, as well as combinations of these organic acid salts. In still another embodiment, the organic acid salts that can be used as an agent include, but are not limited to succinate. In some embodiments, the organic acid salt is a sodium-based salt, a lithium-based or a cyclohexylammonium-based salt. In some embodiments, the organic acid salts can be provided as “pharmaceutically acceptable salts”. As used herein, the term “pharmaceutically acceptable salts” refers to salts of the organic acids that retain the desired biological activity of the active compound and do not impart undesired toxicological effects thereto.

In another embodiment, the agent can be a nucleic acid molecule encoding the hG3PP polypeptide, fragment or variant that is intended to be expressed in a mammalian cell, tissue or subject. In some embodiments, a full length nucleotide sequence encoding the hG3PP polypeptide or a fragment thereof can be used. The nucleic sequence of the nucleic acid molecule that can be used can be derived from the known nucleic acid sequence encoding the mammalian (human) hG3PP (refer, for example, to GenBank Accession Nos. NM_001042371 for the nucleic acid sequence of the human mRNA transcript of the human hG3PP).

A “fragment” of a hG3PP-encoding nucleotide sequence encodes a biologically active portion (e.g. that exhibits phosphatase activity) of the hG3PP polypeptide and will encode at least 15, 25, 30, 50, 75, 100, 125, 150, 175, 200, 225, 250, 275, 300 or more contiguous amino acids. The agent can also be a hG3PP polypeptide variant as disclosed herein.

In addition, the nucleic acid sequences of the nucleic acid molecules can be variants of the hG3PP-encoding nucleotide sequences. “Variants” of the hG3PP-encoding nucleotide sequences include those sequences that encode the hG3PP polypeptide disclosed herein but that differ conservatively because of the degeneracy of the genetic code. These naturally occurring allelic variants can be identified with the use of well-known molecular biology techniques, such as polymerase chain reaction (PCR) and hybridization techniques. Variant nucleotide sequences also include synthetically derived nucleotide sequences that have been generated, for example, by using site-directed mutagenesis but which still encode an hG3PP polypeptide having phosphatase activity. Generally, nucleotide sequence variants have at least 45%, 55%, 65%, 75%, 85%, 95%, or 98% identity to a particular nucleotide sequence disclosed herein. The percentage of identity between the variant sequence and the native sequence can be determined over the entire length of the native sequence. It will be appreciated by those skilled in the art that DNA sequence polymorphisms that lead to changes in the amino acid sequences of the hG3PP polypeptide may exist within a population (e.g., the human population). Such genetic polymorphism in the hG3PP gene may exist among individuals within a population due to natural allelic variation. Any and all such nucleotide variations and resulting amino acid polymorphisms or variations in the sequence of the hG3PP gene that are the result of natural allelic variation and that do not alter the functional activity of the hG3PP polypeptide are intended to be used herein.

In addition to naturally-occurring allelic variants of G3PP-encoding sequences that may exist in the population, the skilled artisan will further appreciate that changes can be introduced by mutation into the nucleotide sequences of the invention thereby leading to changes in the amino acid sequence of the encoded the hG3PP polypeptide, without altering the biological activity of the hG3PP polypeptide. Such mutations can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The nucleic acid molecule can include a promoter for allowing the expression of the hG3PP polypeptide. The promoter can be operatively linked to the nucleic acid sequence encoding the hG3PP polypeptide. The promoter can be selected to be inducible or constitutive, tissue- or cell-specific or not, etc. In one embodiment, the promoter can be derived from the cytomegalovirus (e.g., CMV), can be derived from the albumin gene (for expression in the liver), can be derived from the MIP gene (for expression in pancreatic β cells), etc.

In some embodiments, the nucleic acid molecular can include a nucleic acid sequence encoding a non-hG3PP polypeptide. The non-hG3PP polypeptide can be expressed as a fusion protein with the hG3PP polypeptide or independently from the hG3PP polypeptide.

The nucleic acid molecule may be constituted by ribonucleic acids residues, deoxyribonucleic acid residues or a combination of both.

The nucleic acid molecules may be designed as an oligonucleotide. In the context of the present, the term “oligonucleotide” refers to a synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the agents described herein. Oligonucleotides may also include species that include at least some modified base forms. Thus, purines and pyrimidines other than those normally found in nature may be so employed. Similarly, modifications on the furanosyl portions of the nucleotide subunits may also be affected. Examples of such modifications are 2′-O-alkyl- and 2′-halogen-substituted nucleotides. Some non-limiting examples of modifications at the 2′ position of sugar moieties which are useful in the present invention include OH, SH, SCH₃, F, OCH₃, OCN, O(CH₂), NH₂ and O(CH₂)ₙCH₃, where n is from 1 to about 10. Such oligonucleotides are functionally interchangeable with natural oligonucleotides or synthesized oligonucleotides, which have one or more differences from the natural structure. All such analogs are comprehended by this embodiment so long as they function effectively to allow for the expression of the hG3PP polypeptide as described herein.

The nucleic acid molecules may also be included in an expression vector. Expression vectors can be derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence presented herewith in the mammalian cell.

The nucleic acid molecules may be used to achieve gene therapy. Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Embodiments of the viral vectors that can be used include adenovirus- and lentivirus-based expression vectors.

The use of RNA or DNA based viral systems for the delivery of nucleic acids take advantage of highly evolved processes for targeting a virus to specific cells in the body and trafficking the viral payload to the nucleus. Viral vectors can be administered directly to mammalian subjects (in vivo) or they can be used to treat cells in vitro and the modified cells then administered to mammalian subjects (ex vivo). Conventional viral based systems for the delivery of nucleic acids could include retroviral, lentiviral, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In applications where transient expression of the nucleic acid molecule is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acid molecules, e.g., in the in vitro production of nucleic acids and peptides, and for in vivo and ex vivo gene therapy procedures.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials.

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system.

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defective vector is propagated in human 293 cells that supply the deleted gene function in trans. Ad vectors can transduce multiple types of tissues in vivo, including non-dividing, differentiated cells such as those found in the liver, kidney and muscle tissues. Conventional Ad vectors have a large carrying capacity.

In many gene therapy applications, it is desirable that the gene therapy vector be delivered with a high degree of specificity to a particular tissue type (such as the pancreas). A viral vector is typically modified to have specificity for a given cell type by expressing a ligand as a fusion protein with a viral coat protein on the viruses outer surface. The ligand is chosen to have affinity for a receptor known to be present on the cell type of interest.

Gene therapy vectors can be delivered in vivo by administration to an individual subject, typically by systemic administration (e.g., intravenous, intraperitoneal, intramuscular, subdermal, or intracranial infusion) or topical application. Alternatively, vectors can be delivered to cells ex vivo, such as cells explanted from an individual subject or universal donor hematopoietic stem cells, followed by re-implantation of the cells into the mammalian subject, usually after selection for cells which have incorporated the vector.

Ex vivo cell transfection for gene therapy (e.g. via re-infusion of the transfected cells into the host organism) is well known to those of skill in the art. In a preferred embodiment, cells are isolated from the subject organism, a nucleic acid molecule (gene or cDNA) of interest is introduced therein, and the cells are re-infused back into the mammalian subject. Various cell types suitable for ex vivo treatment are well known to those of skill in the art. In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft at an appropriate location (such as in the bone marrow).

The present invention will be more readily understood by referring to the following examples which are given to illustrate the invention rather than to limit its scope.

### EXAMPLE I

### Characterization of the Role of Mammalian Glycerol-3-Phosphate Phosphatase (G3PP)

Animals. All procedures were approved by the Institutional Committee for the Protection of Animals. Five-week-old male C57BL/6N mice and Wistar rats (85-250 g) were housed on a 12-h light/dark cycle with free access to water and standard diet (15% fat by energy). Mice were fed with either chow or high fat diet (HFD; 60% calories from fat) for 8 weeks. G3PP expression was evaluated in chow fed, HFD fed and overnight fasted mice. To evaluate the effect of feeding and fasting on glycerol-3-phosphate phosphatase (G3PP) expression, ad libitum fed and overnight fasted mice were sacrificed and different tissues were collected for analyses.

Islet and hepatocyte isolation. Pancreatic islets were isolated from rats and mice (Peyot et al., 2009). Isolated islets were cultured overnight at 37° C. in complete RPMI 1640 medium. Hepatocytes were isolated from rats by in situ collagenase perfusion and were seeded in DMEM complete medium (Merlen et al., 2014).

Insulin secretion. Insulin secretion in INS832/13 cells (Hohmeier et al., 2000) and isolated islets was measured in static incubations (Peyot et al., 2009). INS832/13 cells were cultured in 12-well plate for 36 h in complete RPMI medium with 11 mM glucose, and then transferred to medium containing 2 mM glucose for 2 h. Then the cells were washed in Krebs Ringer buffer-Hepes (KRBH) containing 2 mM glucose, 2 mM glutamine, 50 μM carnitine and 0.5% defatted BSA (KRBH 2G/0.5% BSA) and pre-incubated for 45 min in KRBH 2G/0.5% BSA. This was followed by static incubations for 1 h in KRBH with 0.5% defatted BSA, containing glutamine, carnitine and various concentrations of glucose, for measuring insulin release. For insulin secretion from islets, batches of 100 islets were starved 45 min in KRBH medium with 4 mM glucose, 2 mM glutamine, 50 μM carnitine and 0.5% defatted BSA and then washed in KRBH containing 4 mM glucose and 0.5% defatted BSA (KRBH 4G/0.5% BSA), and pre-incubated for 45 min in KRBH 4G/0.5% BSA. Islets were then incubated for 1 h in KRBH with 0.5% defatted BSA containing glutamine, carnitine and different concentrations of glucose, to measure insulin release. At the end of the incubations, media were collected and proteins were extracted from cells or islets. Insulin released into medium was determined by AlphaLISA™ assay (PerkinElmer).

Over-expression and RNAi knockdown of G3PP. The pCMV-based plasmids (Origene) expressing human G3PP (PGP, phosphoglycolate phosphatase; SC311252) and Green Fluorescent Protein (GFP; PS100010) were introduced into INS832/13 cells using the lipofectamine (Life Technologies). After transfection, cells were cultured for 48 h in 96-well, 12-well or 6-well plates. Silencer select pre-designed siRNA against G3PP (rat PGP) and two scrambled-siRNA were obtained from Ambion (G3PP: s220489; scrambled-siRNA-1: 4390844 and scrambled-si-RNAi-2: 4390847). The sequences for G3PP used were 5′-AGGCGGACAUCAUCGGGAAtt-3′ (SEQ ID NO: 1) and 5′-UUCCCGAUGAUGUCCGCCUgg-3′ (SEQ ID NO: 2). siRNA constructs were introduced into INS832/13 cells by reverse transfection using RNAiMAX and used 48 h after transfection (Zhao et al., 2014). Transfected cells were used for Western blotting and measurements of insulin secretion, caspase activity, glycerol and free fatty acids (FFA) release, oxygen consumption and fatty acid oxidation and esterification.

Quantitative real-time PCR. Total RNA was extracted from INS 832/13 cells, islets and rodent tissues using the Rneasy™ Mini kit (Qiagen) with Rnase-free Dnase (Qiagen). First strand cDNA was synthesized from 2 μg of total RNA in 50 μl (final volume) of a buffer containing the Pd(N)₆ random primers and MMLV reverse transcriptase. RT-qPCR was performed using a Rotor-Gene 3000 (Corbett Robotics) and the PCR products were quantified using the FastStart™ DNA Master PLUS SYBR™ green kit (Roche Diagnostics) according to the manufacturer's instructions. Expression levels were normalized for the 18S or cyclophilin mRNA transcript. The primer sequences were: 18S mRNA forward (5′-CTGAGAAACGGCTACCACATC-3′ (SEQ ID NO: 3)), reverse (5′-GGCCTCGAAAGAGTCCTGTAT-3′ (SEQ ID NO: 4)); Cyclophilin forward (5′-CTTGCTGCAGACATGGTCAAC-3′ (SEQ ID NO: 5)), reverse (5′-GCCATTATGGCGTGTGAAGTC-3′ (SEQ ID NO: 6)) mouse G3Pase forward (5′-CTGGACACAGACATCCTCCTG-3′ (SEQ ID NO: 7)), reverse (5′-TCACTTTCCTGATTGCTCTTCA-3′ (SEQ ID NO: 8)); and rat G3Pase forward (5′-GACAACCTCCACTCACTCTGC-3′ (SEQ ID NO: 9)), reverse (5′-AAGTTTAGCTGGGCTGCTGTT -3′ (SEQ ID NO: 10)).

Free fatty acids and glycerol release. For FFA determinations, isolated rat islets were pre-incubated for 1 h in KRBH containing 2 mM glutamine and at 4 mM glucose, 50 μM carnitine and then incubated for 2 h at 4, 10, 16 and 25 mM glucose. The low and high glucose concentrations were 2 and 10 mM, respectively, when INS832/13 cells were used. Incubations were done without and with the panlipase inhibitor, orlistat. Cells and islets were harvested after incubations for protein extraction. FFA released into the medium were extracted by a modified Dole-Meinertz extraction procedure (Puttmann et al., 1993). Briefly, the media (0.5 ml), in Pyrex glass tubes, were mixed with the internal standard [²H₃₁]-palmitic acid and extracted with 2.5 ml of a solvent mixture containing isopropanol-n-heptane-2M phosphoric acid (40:10:1, v/v). After thorough mixing by vortex, the tubes were kept in a bath sonicator (Branson Ultrasonic) and sonicated for 2 min with 30 s intervals, avoiding sample heating. After rigorous mixing by vortex, the tubes were let stand at room temperature for 10 min, and then heptane (1 ml) and water (1.5 ml) were added and the tubes were thoroughly mixed by vortex followed by sonication for 1 min. Then the tubes were centrifuged at 1000×g for 10 min at 4° C. An aliquot of 1.5 ml (88% of the total organic layer) from the top layer was transferred to 2.0 ml reacti-vials™ (Supelco) and dried under nitrogen (N-Evap; Organomation, Berlin, Mass.). The dried fatty acids were derivatized with phenacylbromide and quantified by reverse phase HPLC (Mehta et al., 1998) using a Zorbax Eclipse plus XDB analytical C18 column (4.6×250 mm; 5 μm; Agilent Technology). FFA were eluted using methanol/water (92.5:7.5, v/v) at a flow rate of 1.5 ml/min and absorbance of eluting FFA-phenacyl derivatives was measured at 242 and 254 nm. The peaks were identified by their retention times, and the concentrations of individual fatty acids were computed by the internal standard method from peak area using the standard curves of individual FFA. Glycerol release was determined by a radiometric glycerol assay (Bradley and Kaslow, 1989) using [Γ-³²P]ATP and glycerokinase.

Plasma chemistry. Triglycerides and glycerol concentrations were measured using a quantitative enzymatic colorimetric assay kit (Sigma). Blood glucose and insulin levels were measured as described in Zhao et al., 2014. Plasma low density lipoprotein and high density lipoproteins were assessed using a quantitative enzymatic colorimetric assay kit (Wako, L-Type LDL-C) (Friedewald et al., 1972).

Fatty acid esterification and oxidation. INS832/13 cells were transfected with plasmids expressing hG3PP or GFP (control) or siRNA targeting G3PP or control siRNA, as described above. Fatty acid (FA) esterification and oxidation in the transfected INS832/13 cells were measured after incubating the cells as for insulin secretion experiments, as described above. Then the cells were incubated in KRBH containing 0.1 μCi/ml [1-¹⁴C]palmitate (57.5 mCi/mmol; Amersham Biosciences), 0.1 mM unlabeled palmitate plus 0.5% defatted BSA (for FA oxidation) or 0.2 mM unlabeled palmitate plus 0.5% defatted BSA (for FA esterification), 1 mM carnitine, and 2 or 10 mM glucose. Primary hepatocytes (0.2 million cells/well in 12-well plate for FA oxidation and 0.5 million cells/well in 6-well plate for FA esterification) were first preincubated for 45 min in 1 ml of DMEM containing 5 mM glucose. They were then incubated for 2 h in 0.5 ml (FA oxidation) or 1 ml (FA esterification) of DMEM with 0.25% defatted BSA, 0.1 mM palmitate (oxidation) or 0.2 mM palmitate (esterification), 2 μCi/mI [9,10-³H]palmitate (51 Ci/mmol, Amersham Biosciences), at 5 or 25 mM glucose. For FA esterification determination, at the end of the incubations INS832/13 cells or primary hepatocytes were collected and washed in cold PBS. Total lipids were extracted using Folch reagent (Segall et al., 1999) and separated by TLC using a solvent system (petroleum ether/ether/acetic acid; 70/30/1) to measure the incorporation of labeled palmitate into various lipids species (Nolan et al., 2006). FA oxidation was determined, by collecting and measuring the released ³H₂O into incubation medium (Saddik and Lopaschuk, 1991).

Glucotoxicity and glucolipotoxicity. The effect of either overexpression or knockdown of G3PP in INS832/13 cells on glucotoxicity and glucolipotoxicity was assessed by culturing the transfected cells in 96-well plate for 48 h, followed by replacing the medium with 50 μl of RPMI 1640 medium supplemented with 1% fetal bovine serum, 0.5% BSA and either 5 or 20 mM glucose with or without 0.3 mM palmitate. Incubations were continued for an additional 24 or 72 h after which caspase-3 activity, which reflects apoptosis, was measured. Incubations with 5 mM glucose in the absence of palmitate served as controls. Total caspase-3 activity in each well was determined by using the Caspase 3/7 Luminescent Assay kit (Caspase-Glo, Promega, Madison, Wis.) and normalized to the DNA content in each well, measured with SYBR green I (Molecular Probes) (EI-Assaad et al., 2010).

Oxygen consumption and mitochondrial function. Respiration measurements in vitro were made using a XF24 respirometer (Seahorse Bioscience, Billerica, Mass.). Transfected INS832/13 cells were seeded 48 h before the experiments at 5×10⁴ cells/well in XF24 microplates. Media were changed 2 h before the experiments with complete RPMI 1640 containing 2 mM glucose as described before (Lamontagne et al., 2009). Isolated rat islets after infection with adeno- or lenti-virus constructs, were transferred to XF24 islet capture microplates (75 islets/well) 3 h before the experiments, in RPMI 1640 containing 4 mM glucose, 2 mM glutamine and 50 μM carnitine. Incubations at 37° C. under atmospheric CO₂ were for 1 h for INS cells and 25 min for islets, in KRBH without BSA and bicarbonate. Basal glucose concentration was 2 mM for INS cells and 4 mM for rat islets. After basal respiration measurement for 20 min, glucose levels were elevated to 10 mM (for INS cells) or 16 mM (for islets). After incubations for 20 min or 1 h (for INS cells and islets, respectively), oligomycin, FCCP and antimycin/rotenone were added by three successive injections. The F1F0 ATP synthase inhibitor oligomycin was used to assess uncoupled respiration, FCCP to estimate maximal respiration and antimycin/rotenone to measure non-mitochondrial respiration (Qiang et al., 2012).

Gluconeogenesis and glycolysis in hepatocytes. After infection with adeno- or lenti-viral constructs, primary hepatocytes were starved in DMEM without glucose for 2 h, and then washed with PBS (37° C.), followed by incubation for 2 h in glucose production buffer consisting of glucose-free DMEM (pH 7.4) without phenol red, supplemented with 15 mM Hepes and 2 mM L-glutamine, either with 10 mM glycerol or 20 mM sodium lactate plus 2 mM sodium pyruvate. Then, the medium was processed for glucose measurement (Autokit Glucose Wako). Glycolysis was assessed by measuring lactate production (Phillips et al., 1995). Virus-infected primary hepatocytes were starved as described above and incubated in phenol red-free DMEM (buffered with HEPES pH 7.4), 2 mM L-glutamine and 2 or 25 mM glucose. Lactate accumulated in the cells and released into the medium was measured separately as described before (Maughan, 1982).

G3PP-adenovirus administration to rats. Male Wistar rats (85-100 g; Charles River) were housed in individual cages and given free access to standard diet. Rats received a single injection of adenovirus (5.5×10¹⁰ viral particles/ml/100 g BW) carrying the genes of either human G3PP (Ad-hG3PP) or Green fluorescent protein (Ad-GFP) as control, by tail vein. Rats were given FK506 (0.2 mg/kg body weight) on the day before and on the day of the virus administration to minimize the immune response. Food consumption and body weight were monitored daily for 7 days following virus injection. Then, food was withdrawn for 12 h and glycerol load test performed as described below. Rats were sacrificed and blood was collected by heart puncture and different tissues were collected and were clamp frozen and stored at −80° C. till further analysis.

Adenovirus and lentivirus infection of islets and hepatocytes. Islets and hepatocytes were infected with either recombinant adenoviruses or lentivirus at a multiplicity of infection of 100 or 5 respectively, 48 h before utilization. For islet adenoviral infection, after isolation islets were incubated with Hanks' balanced salt solution (HBSS) containing 5 mM glucose, 1 mM EGTA at 37° C. for 3 min before infecting with recombinant adenoviruses: 200 islets per well in a 6-well plates for overnight at 37° C. Hepatocytes adenoviral infection was made with 0.2 million cells/well in 12-well plates and 0.5 million cells/well in 6-well plates. Both islets and hepatocytes were infected with recombinant adenovirus expressing GFP alone (control) or human G3PP (Vector Biolabs), both under CMV promoter control, to overexpress these proteins. In order to knockdown endogenous G3PP, we used rat G3PP-shRNA lentivirus, with a GFP-shRNA lentivirus as control (abm®). The conditions of infection with lentiviruses were similar as for adenoviruses.

Oral glycerol load test. Rats injected with G3PP or GFP expressing adenovirus were fed chow diet for 1 week and on the 7^{th} day, food was withdrawn for 12 h (from 18:00 until 06:00 the following day) prior to administration of glycerol load. Then, 87% glycerol (5 mg/g BW) was administered orally. Blood was collected from tail vein before glycerol load and blood glucose was measured at 5, 10, 20, 30, 50 and 60 min following glycerol load using an Accu-Chek Sensor glucometer (Roche).

G3PP protein expression and activity in vitro. Recombinant human G3PP was expressed in 293T cells. Total cell extracts were prepared from cells expressing hG3PP and GFP (control). G3PP activity was measured by assaying the release of glycerol, using glycerokinase and Γ³²P-ATP. All tested compounds were from Sigma Aldrich. The reaction mix (final volume, 100 μl) containing 50 mM triethanolamine-HCl (pH 7.5), 200 mM NaCl, 5 mM MgCl₂ and indicated concentrations of glycerol-3-phosphate, was pre-incubated for 10 min at room temperature and the reaction was started by the addition of enzyme source (cell extract). To derive K_{M} and K_{cat} values, the data were fit by nonlinear regression to the Michaelis Menten equation using GraphPad Prism. All phosphatase assays were performed with three independently prepared cell transfections.

Immunoblotting. Tissue and cell lysates were prepared and extracted proteins were processed for immunoblotting (Peyot et al., 2009). Membranes were incubated with antibodies for G3PP/PGP (Santa Cruz Biotechnology, sc-241605, dilution 1:1000). Mouse monoclonal β-actin (dilution 1:10000) and rabbit polyclonal α-tubulin (dilution 1:20000) antibodies were from Sigma and Abcam (Cambridge, Mass.) respectively.

Statistical analysis. Values are expressed as means±SEM. Statistical significance was calculated with one-way or two-way analysis of variance (ANOVA) with Bonferroni or Dunnett's post hoc testing for multiple comparisons or the Student's t test, as indicated. A P value of <0.05 was considered significant.

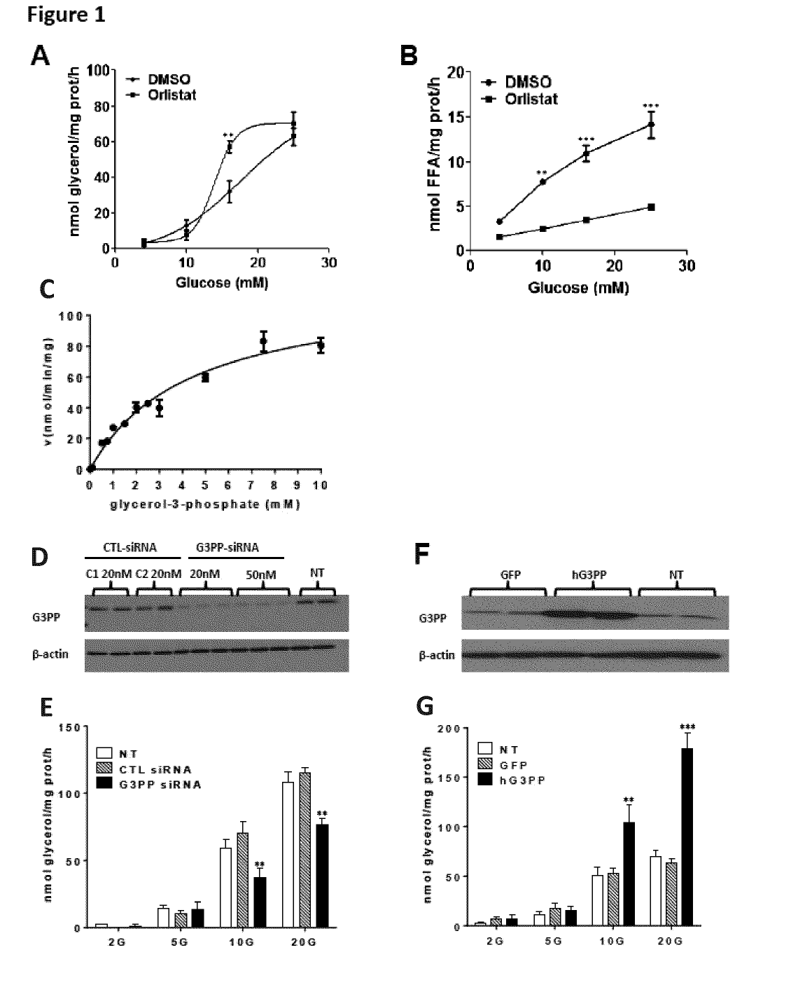

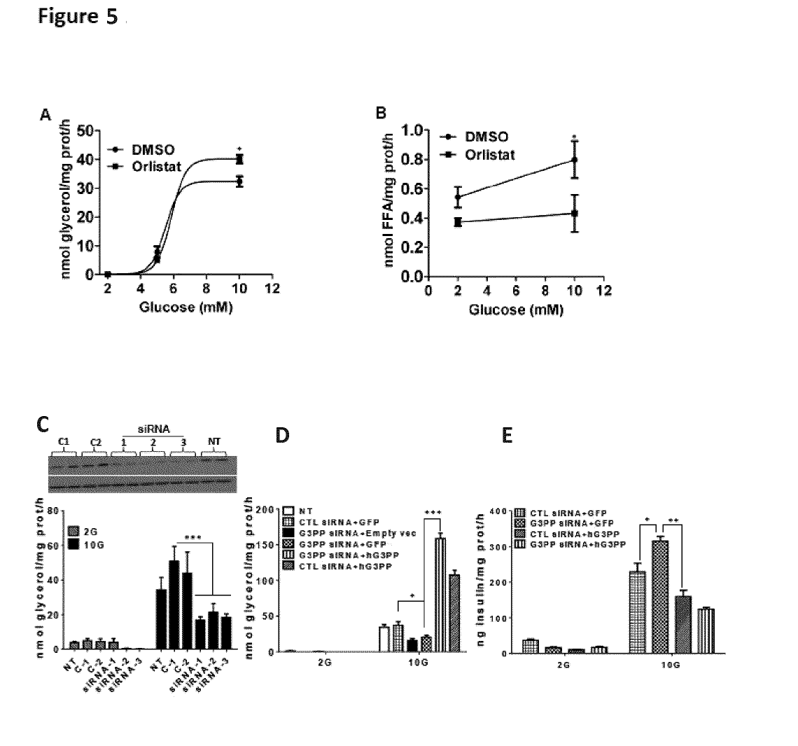

Dichotomy in orlistat effect on glycerol and FFA release in β-cells. The discovery of a mammalian G3PP started from the fortuitous observation of a dichotomy of inhibitory effects of the panlipase and lipolysis inhibitor orlistat on glycerol and FFA release at various glucose concentrations from β-cells. Thus, orlistat inhibited lipolysis at high glucose concentrations in INS832/13 β-cells and in rat islets as evidenced by the reduction in FFA release; however, the increased release of glycerol in the presence of elevated glucose concentration was not inhibited (FIGS. 1A, 1B; FIGS. 5A and 5B), indicating that not all glucose-derived glycerol arises from lipolysis. In rat islets at medium concentration of glucose (10 mM), orlistat showed moderate inhibition of glycerol release indicating that at this glucose concentration, a small amount of glycerol does arise from lipolysis. Thus in β-cells there must exist an alternate mechanism for producing glycerol, besides lipolysis. The direct hydrolysis of glucose-derived Gro3P by a hypothetical Gro3P phosphatase is a plausible source of glycerol.

Phosphoglycolate phosphatase acts as a specific G3PP. Recombinant human PGP showed high activity with glycerol-3-phosphate, with a K_{M} of 1.5 mM and k_{cat} (s⁻¹) of 0.1 (FIG. 1C). High glucose concentration stimulated glycerol release in INS832/13 β-cells was reduced by RNAi-knockdown of native PGP (FIGS. 1E, 1F; FIG. 5C), greatly elevated by overexpression of human PGP (FIGS. 1G and 1H), and the decrease caused by RNAi-knockdown was reversed by overexpression of hPGP in the same cells (FIG. 5D). Overall the data demonstrate that PGP acts as a G3PP in vitro and in intact cells.

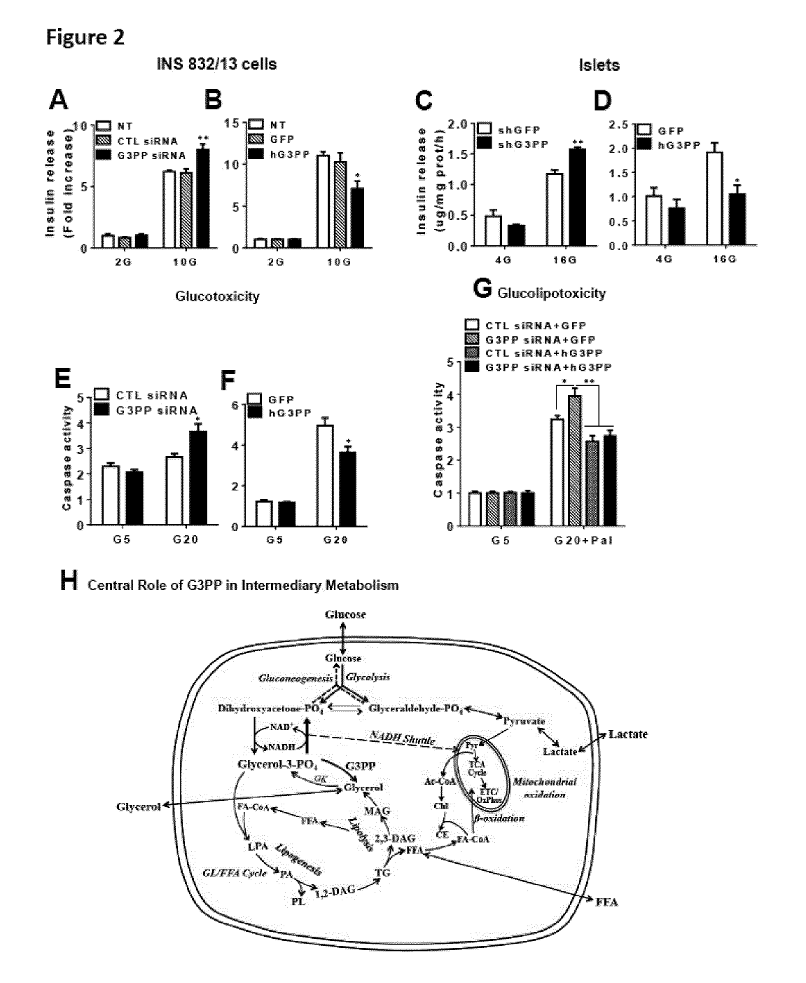

G3PP activity controls insulin secretion and glucolipotoxicity in pancreatic β-cells. Since Gro3P is one of the starting substrates for the GL/FA cycle that produces lipid signals for glucose stimulated insulin secretion (GSIS), alteration of Gro3P levels by G3PP should influence insulin secretion. All the three different G3PP-siRNAs reduced G3PP expression effectively (FIG. 5C) and we selected G3PP-siRNA-1 and control siRNA-1for rest of the study. In accordance with this prediction, RNAi-knockdown of native rat G3PP in INS832/13 β-cells elevated GSIS (FIG. 2A) while overexpression of hG3PP reduced GSIS (FIG. 2B), without affecting basal secretion. Similar results were obtained in isolated rat islets infected with lentiviral shRNA-G3PP for RNAi-knockdown or adenoviral hG3PP for overexpression (FIGS. 2C, 2D). The role of G3PP activity in regulating GSIS was confirmed by the observation that overexpression of hG3PP in INS832/13 cells curtailed the increased GSIS caused by RNAi-knockdown of endogenous G3PP (FIG. 5E).

Chronic elevated glucose exposure of β-cells without or with high concentrations of exogenous FFA cause glucotoxicity and glucolipotoxicity, respectively, as indicated by caspase-3 activity, an index of apoptosis. The mechanism involves enhanced glucose metabolism and esterification of FFA resulting in mitochondrial dysfunction, ROS production and ER stress. Reducing G3PP expression in INS832/13 β-cells, which is likely to elevate the formation of glycerolipid intermediates, caused enhanced glucotoxicity, while overexpression of hG3PP led to decreased glucotoxicity (FIG. 2E). Glucolipotoxicity, which was enhanced by G3PP knockdown, was curtailed by hG3PP overexpression that also reversed the toxic effect of G3PP knockdown under glucolipotoxic condition (FIG. 2F). Thus, changes in G3PP activity in the p-cell modulate insulin secretion and the response to metabolic stress.

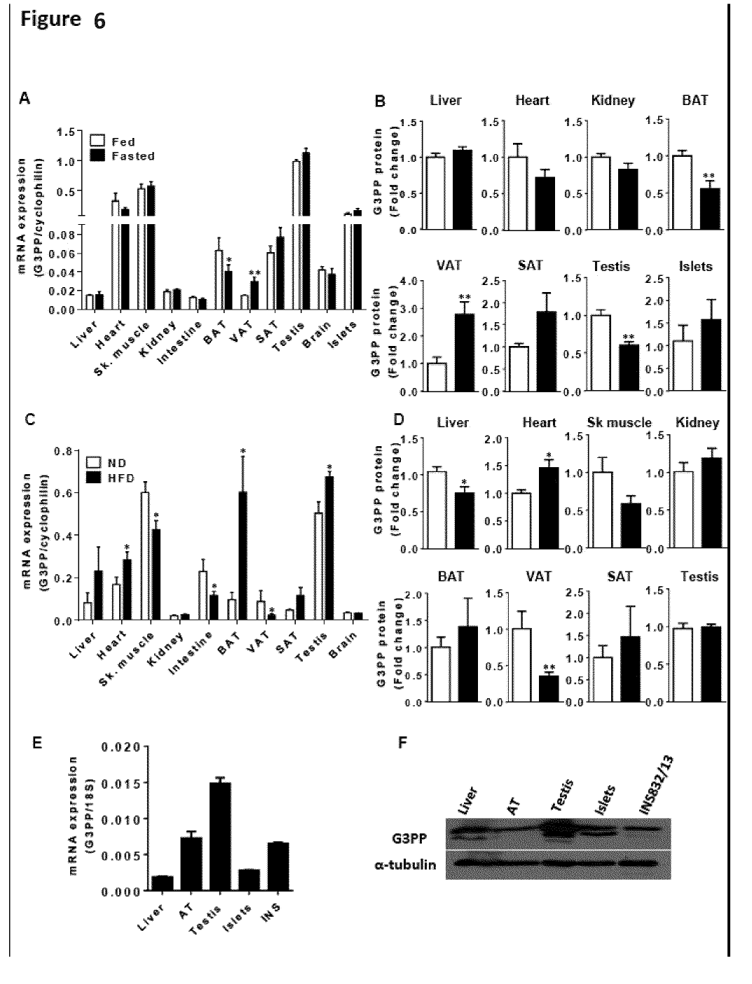

Tissue distribution and nutritional regulation of G3PP. Expression of G3PP both at the mRNA (FIGS. 6A, 6C, 6E) and protein (FIGS. 6B, 6D, 6F) levels is apparently ubiquitous since it was detected in all tissues examined; it was found particularly high in testis followed by heart, skeletal muscle and islet tissue. Liver, kidney, intestine and visceral white adipose tissue showed low expression, probably because these tissues are engaged in either gluconeogenesis and/or lipogenesis, both of which require Gro3P supply. The high expression of G3PP in heart and skeletal muscle possibly ensures no toxic accumulation of lipids in these fat burning tissues. The role of this enzyme in testis is not clear.

G3PP expression is regulated by nutritional status. Thus G3PP mRNA and protein is inversely changed in white adipose vs brown adipose under fed and fasted state (FIGS. 6A, 6B) and under high fat diet (HFD) vs normal diet conditions (FIGS. 6C, 6D). These changes may reflect the adaptation for regulation of nutrient metabolism in adipose tissues. Thus, elevated G3PP in white adipose in fasted state ensures supply of glycerol into circulation rather than glycerol re-incorporation into glycerolipids, for the purposes of gluconeogenesis in liver and kidney, whereas the decreased G3PP expression in brown adipose ensures trapping of incoming FFA and glucose into glycerolipids, for future usage, as well as for fuel usage for thermogenesis during fasting. Conversely, the decreased expression of G3PP in white adipose tissue in HFD condition should help in the storage of fat while in brown adipose, such storage is not needed and the elevated G3PP levels ensure effective burning of fatty acids in brown adipose tissue (BAT) mitochondria. Hence, nutritional control of G3PP exemplifies the importance of this enzyme in fuel and energy metabolism as its expression is differentially regulated in different tissues.

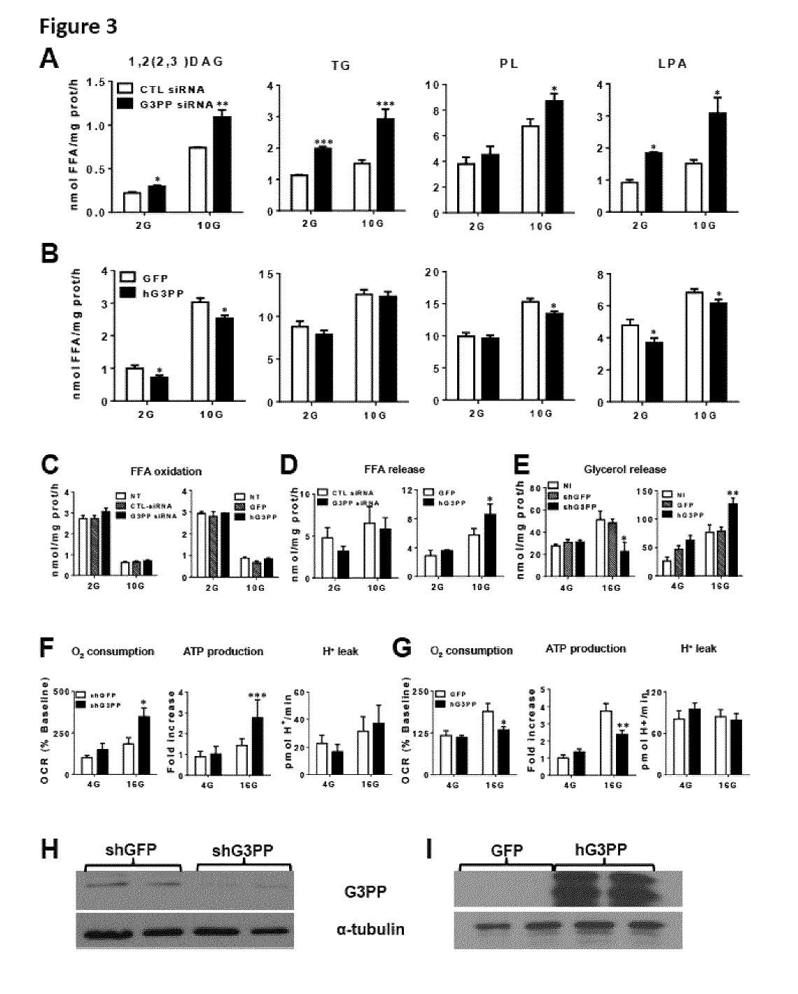

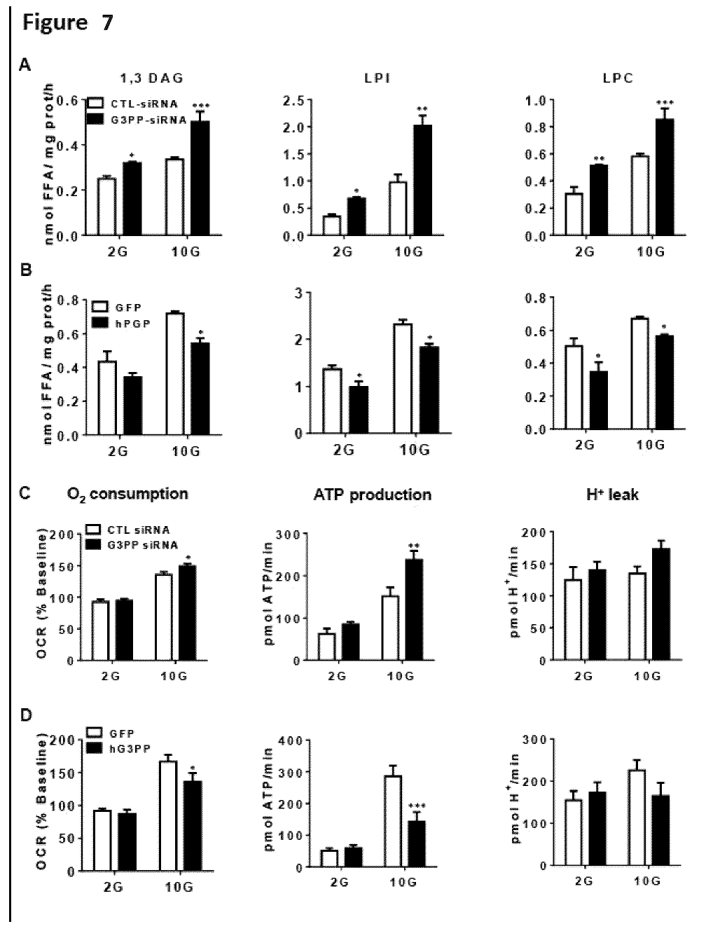

G3PP expression level influences glucose, lipid and energy metabolism in β-cells. As expected, RNAi knockdown of G3PP in INS832/13 cells increased the synthesis of 1,2-DAG, 1,3-DAG, TG, total phospholipids, lysophosphatidylinositol, lysophosphatidate and lysophosphatidylcholine (FIGS. 3A, 7A), whereas overexpression led to their decreased synthesis (FIGS. 3B and 7B). Considering that many of these lipids have signaling roles in different cells, G3PP is likely to regulate these signaling pathways. In INS832/13 cells, altered activity of G3PP had no effect on fatty acid oxidation either at low or high glucose concentration (FIG. 3C). FFA release from these cells, which is mostly dependent on lipolysis, was elevated when G3PP was overexpressed, indicating that a reduction in Gro3P levels following G3PP overexpression, lowers the reesterification of FFA, leading to their elevated release from the cells (FIG. 3D). In rat islets, glucose-stimulated glycerol release was lowered by G3PP knockdown and increased by G3PP overexpression (FIG. 3E), similar to that noticed with INS832/13 cells (FIGS. 1F, 1H).

Since Gro3P directly transfers electrons to mitochondria via the action of mitochondrial Gro3P dehydrogenase, changes in Gro3P levels during glucose oxidation, are expected to influence respiration. Thus, in rat islets reducing G3PP expression led to elevated O₂ consumption and ATP production (FIG. 3F), while hG3PP overexpression caused opposite changes (FIG. 3G), without affecting H⁺ leak in both cases. Similar results were obtained using INS832/13 cells (FIGS. 7C, 7D). Altered G3PP protein levels were confirmed in rat islets after shRNA knockdown and hG3PP overexpression (FIGS. 3H, 3I). The increased ATP levels in β-cells by G3PP knockdown relate to the increased GSIS seen under these conditions. Thus altered expression of G3PP in β-cells has a significant impact on glucose, lipid and mitochondrial metabolism and consequently on the response of these cells for metabolic signal transduction and GSIS.

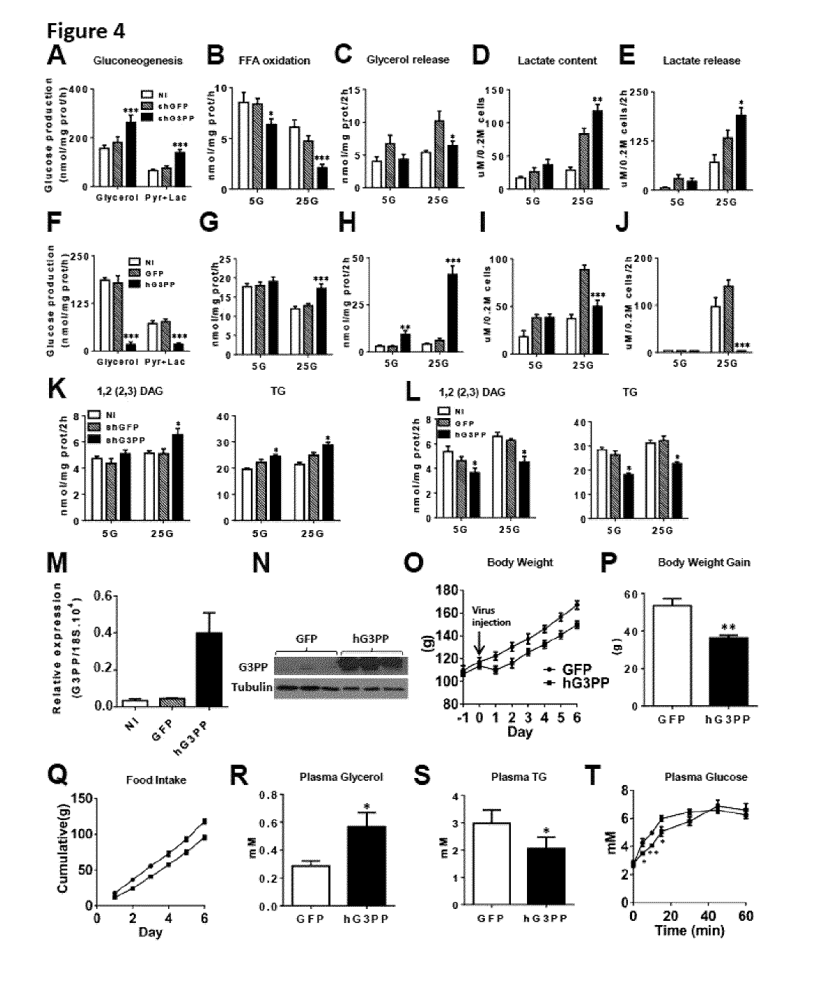

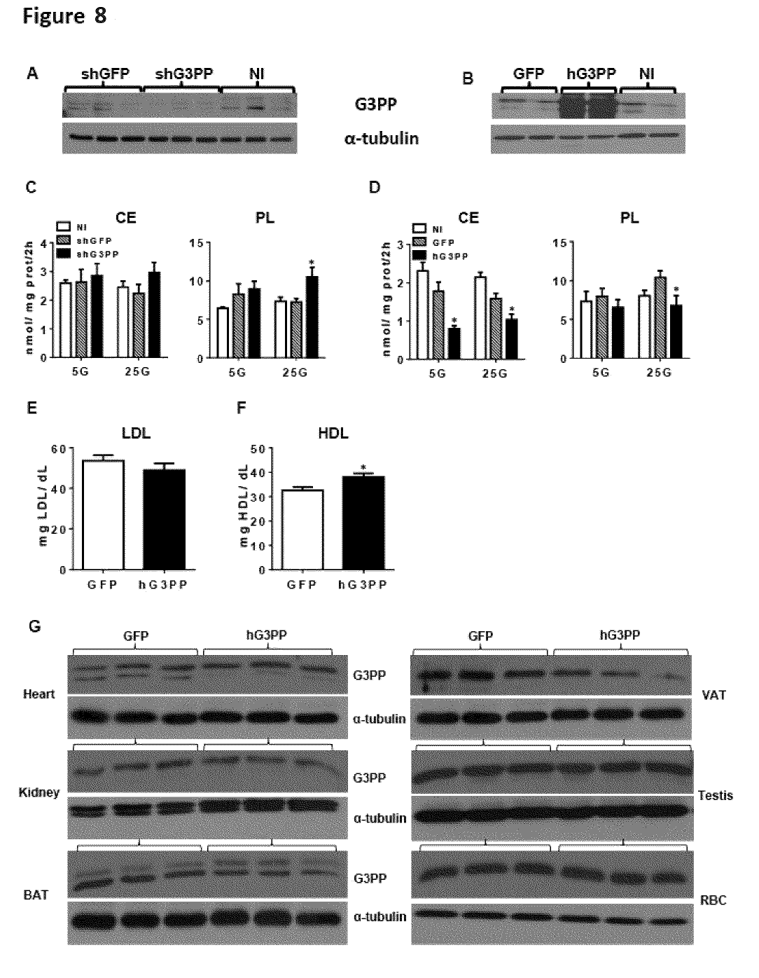

G3PP controls glycolysis, gluconeogenesis and lipid metabolism in hepatocytes. Because Gro3P is a central metabolic intermediate that lies at the crossroads of glucose and lipid metabolism, it was examined whether G3PP also plays a critical role in metabolic regulation in hepatocytes. Liver is the major site of gluconeogenesis starting either from amino acids or adipose lipolysis derived glycerol and both pathways involve the formation of Gro3P. Thus, in primary rat hepatocytes, shRNA-knockdown of G3PP (FIG. 8A) led to a great increase in gluconeogenesis both from glycerol and from pyruvate+lactate (FIG. 4A), whereas overexpression of hG3PP in these cells (FIG. 8B) completely curtailed gluconeogenesis (FIG. 4F).

Fatty acid oxidation in liver is dependent on the availability of fatty acyl-CoA substrate, which is controlled by the extent of esterification by glycerol-phosphate acyltransferase-1. Fatty acid oxidation was directly related to G3PP expression levels in rat hepatocytes, and at high glucose, which suppress p-oxidation, elevated G3PP expression caused enhanced fatty acid oxidation (FIGS. 4B, 4G). This is different from the results with INS832/13 cells and probably reflect the highly lipogenic nature of liver tissue as compared to β-cells. Thus, FFA entering cells must be esterified before being oxidized following lipolysis of endogenous lipid stores. In hepatocytes also, glycerol release at high glucose was reduced by G3PP knockdown and elevated by its overexpression (FIGS. 4C, 4H).

Knockdown of G3PP in hepatocytes enhanced lactate production and release, an index of glycolytic flux, as expected, because of decreased diversion of glucose carbons in the form of glycerol via G3PP (FIGS. 4D, 4E). Conversely, overexpression of hG3PP had reverse effects, reducing glycolytic flux (FIGS. 4I, 4J). The overall increase in glycolytic flux compared to non-infected cells is due to viral infection, which is known to accelerate glycolysis. Similar to the changes in INS832/13 cells, lipogenesis was affected by altered G3PP expression in rat hepatocytes (FIGS. 4K, 4L, 8C and 8D). Formation of cholesterol esters was markedly decreased by the overexpression of G3PP in liver cells (FIG. 8D), and this may be due to reduced availability of fatty acyl groups due to their enhanced flux through mitochondrial β-oxidation.

In vivo overexpression of G3PP reduces hepatic glucose production and plasma triglycerides in rats. In order to further understand the metabolic regulatory role of G3PP, an adenoviral vector coding for hG3PP or GFP (control) was injected to rats. One week post injection, expression of G3PP in liver was greatly elevated (FIGS. 4M, 4N) while it was not altered in other tissues (FIG. 8). One day post injection Adv-hG3PP injected rats showed −10% reduction in body weight, which was maintained for the next 6 days as compared to Adv-GFP injected rats (FIGS. 40, 4P). Adv-G3PP rats also showed a modest reduction in cumulative food intake (FIG. 4Q). After one week, plasma glycerol levels were markedly elevated (FIG. 4R), indicating that the overexpressed G3PP in liver is able to generate glycerol in vivo, which is released into blood. In agreement with the observation in isolated hepatocytes and INS832/13 cells, in vivo liver overexpression of G3PP led to reduced plasma TG levels (FIG. 4S), which is likely due to reduced hepatic TG synthesis. Circulating low-density and high-density lipoproteins (LDL and HDL) were modestly affected, with HDL showing a significant increase (FIGS. 8E, 8F) and LDL a trend of decrease. Hepatic glucose production from glycerol during a glycerol load test was reduced in Adv-G3PP injected rats (FIG. 4T), showing that liver gluconeogenesis from glycerol was affected, as was the case with isolated rat hepatocytes.

The possibility of Gro3P hydrolysis in mammalian cells and fish was previously considered. It has been reported that preparations of fish liver (Ditlecadet and Driedzic, 2013), rat heart (De Groot et al., 1994), and rat brain (Nguyen et al., 2007), can generate glycerol and inorganic phosphate from Gro3P. However, the molecular identity of the mammalian enzyme(s) responsible for this catalytic activity, and their physiological significance, are unknown. In a recent work, it has been suggested that in liver there is a NADH/NAD⁺ ratio dependent direct formation of glycerol from Gro3P, generated by high carbohydrate intake, particularly under conditions of mitochondrial aspartate-glutamate carrier isoform-2 (citrin) deficiency; however no enzyme for this conversion was suggested (Moriyama et al., 2015).

In sum, a metabolic enzyme in mammalian cells that can directly transform Gro3P to glycerol was identified. The identification of a previously unrecognized G3PP in mammalian cells is an important addition to our understanding of metabolic regulation and signaling at large. We have shown that G3PP expression level controls several metabolic pathways and biological processes in a tumoral β-cell line and in normal rat islets as well as in hepatocytes. These include, depending on the cell type, glycolysis, gluconeogenesis, lipogenesis, phospholipid synthesis, lipolysis, fatty acid oxidation and mitochondrial energy metabolism and ATP production. In addition G3PP regulates glucose induced insulin secretion and the response to metabolic stress in the β-cell. Thus G3PP is an attractive target for metabolic syndrome related disorders. It is anticipated that enhanced activity of G3PP to be beneficial under conditions of type-2 diabetes and obesity, as it protects β-cells from fuel surfeit toxicity and from exhaustion due to over-stimulation by high glucose concentrations and reduces hepatic glucose production and lipogenic burden.

### EXAMPLE II

### Effect of Different Organic Acids on G3PP Activity

In order to examine the effect of various compounds on G3PP activity, human G3PP (hG3PP) was overexpressed in 293T cells, using a plasmid vector. Simultaneously control cells received GFP (Green Fluorescent Protein) expression plasmid. After 72 h following transfection, cells were harvested and whole cell extracts were prepared in Krebs Ringer Buffer (KRBH). G3PP was assayed using 104 G3PP cell extract protein (or GFP control extract) with 10 mM Gro3P substrate and 2 or 10 mM test organic acid (R-hydroxy butyric acid, glycolate, succinate, citrate, malate, fumarate, lactate and malonate). Incubations were for 30 min at 30° C. Then the reactions were stopped by adding perchloric acid and glycerol released was measured using Γ-³²P-labeled ATP plus glycerokinase. The amount of glycerol release is proportional to the activity of hG3PP in the cell extracts. GFP cell extracts do not have any significant G3PP activity over reagent blank.

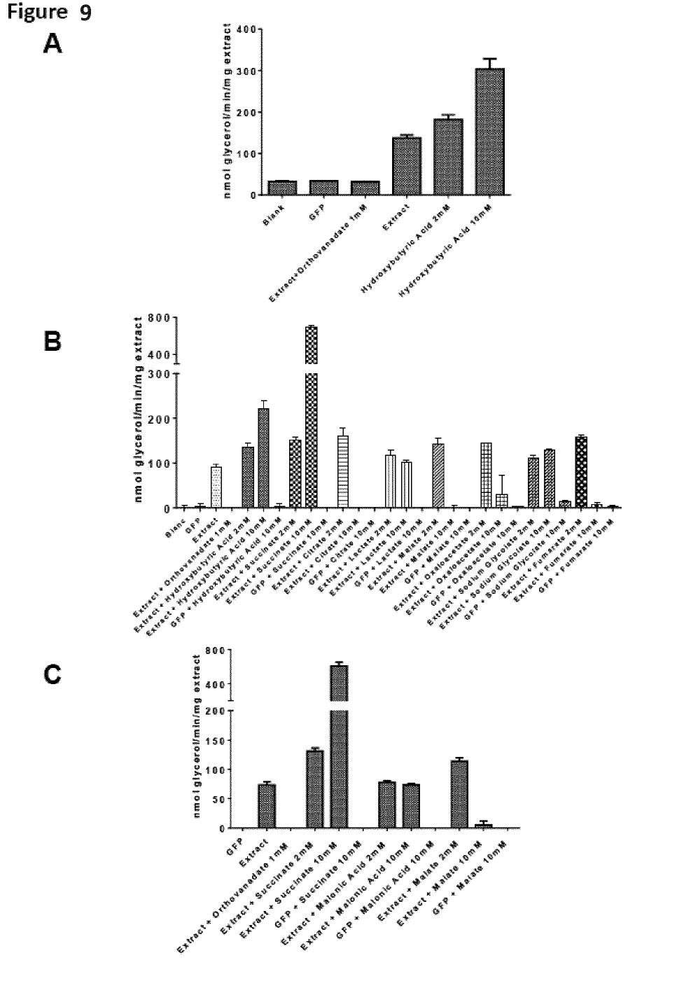

In this assay, glycerol-3-phosphate's concentration is saturating at 10 mM. As such, the activating effect is likely due to the acid's effect on Vₘₐₓ than on K_{M}. Further, the pH is the assay is controlled. As shown on FIGS. 9B and 9C, succinate achieves a marked stimulation of G3PP's activity. The observed effect was marked for succinate as closely related acids such as malonate, malate and fumarate do not activate similarly. Overall, these results show that G3PP's activity can be stimulated by some organic acids (with or without hydroxyl groups, such as, for example, beta-hydroxybutyrate or succinate).

### EXAMPLE III

### Elevated G3PP Levels Reduce Cancer Cell Survival and Proliferation

In order to examine the effect of elevated expression of hG3PP on the survival and proliferation of cancer cells, hG3PP was overexpressed in different human cancer cells (e.g., A549, human alveolar adenocarcinoma cell line; HeLa, human cervical cancer cell line; PC3, human prostatic adenocarcinoma cell line) using a adenoviral-hG3PP vector. In parallel, the controls were set up where Green Fluorescent Protein was overexpressed in cancer cells using adenoviral-GFP vector. The following procedure is used.

Cells were grown for at least one passage in the recommended media adjusted to 10 mM glucose. All media and the FBS were purchased from Life Technologies. A549 cells were grown in DMEM 10 mM Glucose 5% FBS; HeLa cells were grown in DMEM 10 mM Glucose 10% FBS; PC3 cells were grown in RPMI 10 mM Glucose 10% FBS.

For cell passaging, T-75 flasks at 70-80% confluency of cells were rinsed with warmed, sterile PBS and treated with either 2 mL trypsin (A549, HeLa) or 3 mL trypsin (PC3) for 3-5 minutes. Once the cells detached, trypsin was inhibited with 13 or 12 mL of medium, to yield a 15 mL cell suspension. Serial passages were performed by adding 1 or 3 mL of cell suspension to a total of 15 mL of media.

Cells were counted using a hemacytometer, and exactly the same number of cells (3×10⁵ cells for A549 and PC3; 1.5×10⁵ cells for HeLa) per well were seeded for each cell type. Extra medium was added to bring the volume in each well up to 2 mL. Cells were grown in 6 well plate (Corning) for 24 h (70% confluency), then treated with adenoviral-hG3PP or adenoviral-GFP vectors (MOI=100/cell). The virus was left on the cells for 24 h, then removed and replaced with 2 mL of fresh media. Cells were further incubated for 48 h before being harvested and counted again. The total overexpression time was 72 h.

For harvesting after infection, the spent media was collected in a 15 mL conical tube, the cells were washed with 1 mL of sterile, warmed PBS which was also recovered for counting, and trypsin was added at 0.5 mL per well. The plate was incubated for 3 to 5 minutes, and the wells washed with 1 mL of media and collected with cells. All the solutions containing cells were centrifuged at 1200 rpm for 2 minutes, and the supernatant was removed. The pellets containing the cells were resuspened in 1 mL of warm PBS, 100 μL cell suspension was mixed with 100 μL of Trypan Blue vital stain, and counted with a hemacytometer, with both the number of live and dead cells recorded.

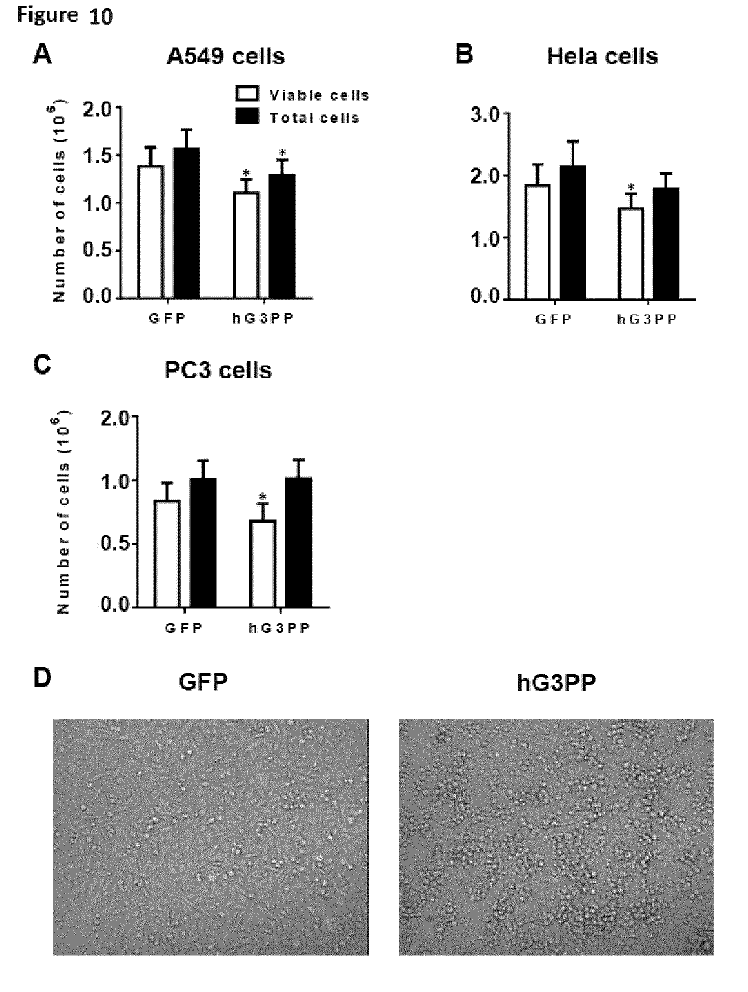

As shown in FIG. 10, there was a marked reduction in hG3PP overexpressing cancer cell viability and proliferation as detected by total number of cells, in comparison to GFP overexpressing corresponding cancer cells This was the case with A549 lung cancer cells (FIG. 10A), HeLa cervical cancer cells (FIG. 10B) and also the PC3 prostate cancer cells (FIG. 10C). There were many floating dead cells when the cells were overexpressing hG3PP, as compared to GFP overexpressing cells. An example of this was shown with PC3 cells in FIG. 10D. These results emphasize that elevated activity of G3PP in cancer cells reduces their proliferation and survival and thus compounds activating G3PP can have potential anti-cancer effects. Pancreatic cancer cells (MiaPaCa2 and PANC1 cell lines) were infected with hG3PP expressing or green fluorescent protein (GFP control) adenoviral vectors and cultured for 48 h in 10 mM glucose and 0.5 mM glutamine containing medium. Then proliferation, apoptosis, DNA content and lactate production (index of glycolysis) were measured. Apoptosis was measured by assaying caspase activity using a fluorescent substrate, in pancreatic cancer cells overexpressing either G3PP or GFP in 96 well plates. In the same wells DNA content was measured using SybrGreen™ method. Caspase activity was normalized to DNA content. Cancer cell proliferation was measured by assaying DNA content of the cells with SybrGreen™ and also by following mitochondrial dehydrogenase activity. Lactate was measured by using an enzymatic method. All results are mean±SEM.

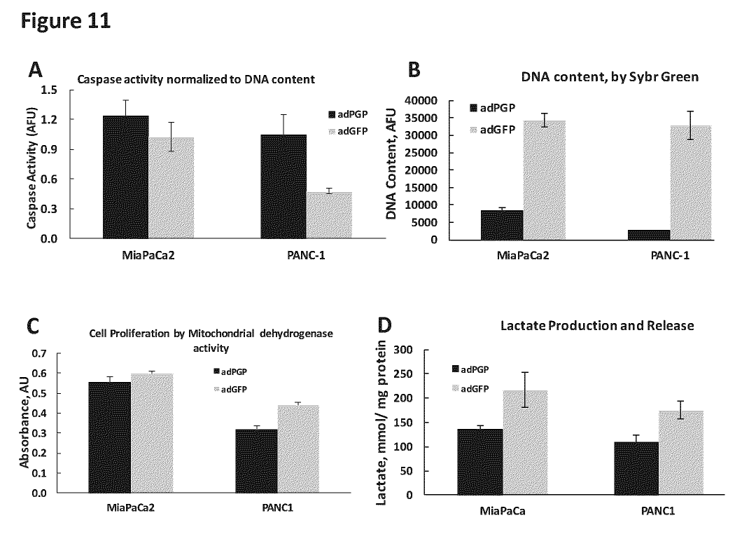

As shown in FIG. 11, overexpression of G3PP in pancreatic cancer cells causes increased apoptosis (FIG. 11A), decreased proliferation (FIGS. 11B and 11C) and reduced glycolysis (FIG. 11D), which is essential for cancer cell growth.

Glycolysis is an important cellular activity of glucose utilization on which many types of cancer cells are dependent to derive energy and cellular building blocks. Lactate is the end-product of glycolysis in cancer cells and it is an index of glycolytic activity. Glycerol-3-phosphate is an important intermediate byproduct of glycolysis, which is essential for lipid synthesis. Lipids are essential for membrane formation and thus cell proliferation. Without wishing to be bound to theory, overexpression of G3PP in pancreatic cancer cells seems to break down glycerol-3-phosphate and blocks glycolysis and ultimately resulting in inhibition of cancer cell growth and survival. Overexpression of G3PP inhibited lactate production from glucose in cancer cells (FIG. 11D).

While the invention has been described in connection with specific embodiments thereof, it will be understood that the scope of the claims should not be limited by the preferred embodiments set forth in the examples, but should be given the broadest interpretation consistent with the description as a whole.

### REFERENCES

de Groot M J, de Jong Y F, Coumans W A, van der Vusse G J. The hydrolysis of glycerol-3-phosphate into glycerol in cardiac tissue: possible consequences for the validity of glycerol release as a measure of lipolysis. Pflugers Arch. 1994 May;427(1-2):96-101.

Ditlecadet, D., and Driedzic, W. R. (2013). Glycerol-3-phosphatase and not lipid recycling is the primary pathway in the accumulation of high concentrations of glycerol in rainbow smelt (Osmerus mordax). Am. J. Physiol. Regul. Integr. Comp. Physiol. 304, R304-312.

El-Assaad, W., Joly, E., Barbeau, A., Sladek, R., Buteau, J., Maestre, I., Pepin, E., Zhao, S., Iglesias, J., Roche, E., et al. (2010). Glucolipotoxicity alters lipid partitioning and causes mitochondrial dysfunction, cholesterol, and ceramide deposition and reactive oxygen species production in INS832/13 ss-cells. Endocrinology 151, 3061-3073.

Friedewald, W. T., Levy, R. I., and Fredrickson, D. S. (1972). Estimation of the concentration of low-density lipoprotein cholesterol in plasma, without use of the preparative ultracentrifuge. Clinical chemistry 18, 499-502.

Hohmeier, H. E., Mulder, H., Chen, G., Henkel-Rieger, R., Prentki, M., and Newgard, C. B. (2000). Isolation of INS-1-derived cell lines with robust ATP-sensitive K+ channel-dependent and -independent glucose-stimulated insulin secretion. Diabetes 49, 424-430.

Lamontagne, J., Pepin, E., Peyot, M. L., Joly, E., Ruderman, N. B., Poitout, V., Madiraju, S. R., Nolan, C. J., and Prentki, M. (2009). Pioglitazone acutely reduces insulin secretion and causes metabolic deceleration of the pancreatic beta-cell at submaximal glucose concentrations. Endocrinology 150, 3465-3474.

Maughan, R. J. (1982). A simple, rapid method for the determination of glucose, lactate, pyruvate, alanine, 3-hydroxybutyrate and acetoacetate on a single 20-mul blood sample. Clinica chimica acta; international journal of clinical chemistry 122, 231-240.

Merlen, G., Gentric, G., Celton-Morizur, S., Foretz, M., Guidotti, J. E., Fauveau, V., Leclerc, J., Viollet, B., and Desdouets, C. (2014). AMPKalpha1 controls hepatocyte proliferation independently of energy balance by regulating Cyclin A2 expression. Journal of hepatology 60, 152-159.

Moriyama M, Fujimoto Y, Rikimaru S, Ushikai M, Kuroda E, Kawabe K, Takano K, Asakawa A, Inui A, Eto K, Kadowaki T, Sinasac D S, Okano Y, Yazaki M, Ikeda S I, Zhang C, Song Y Z, Sakamoto O, Kure S, Mitsubuchi H, Endo F, Horiuchi M, Nakamura Y, Yamamura K I, Saheki T. Mechanism for increased hepatic glycerol synthesis in the citrin/mitochondrial glycerol-3-phosphate dehydrogenase double-knockout mouse: Urine glycerol and glycerol 3-phosphate as potential diagnostic markers of human citrin deficiency. Biochim Biophys Acta. 2015 May 5;1852(9):1787-1795.

Nguyen N H, Gonzalez S V, Hassel B. Formation of glycerol from glucose in rat brain and cultured brain cells. Augmentation with kainate or ischemia. J Neurochem. 2007 June;101(6):1694-700. Epub 2007 Feb 5.

Nolan, C. J., Leahy, J. L., Delghingaro-Augusto, V., Moibi, J., Soni, K., Peyot, M. L., Fortier, M., Guay, C., Lamontagne, J., Barbeau, A., et al. (2006). Beta cell compensation for insulin resistance in Zucker fatty rats: increased lipolysis and fatty acid signalling. Diabetologia 49, 2120-2130.

Peyot, M. L., Pepin, E., Lamontagne, J., Latour, M. G., Zarrouki, B., Lussier, R., Pineda, M., Jetton, T. L., Madiraju, S. R., Joly, E., et al. (2010). Beta-cell failure in diet-induced obese mice stratified according to body weight gain: secretory dysfunction and altered islet lipid metabolism without steatosis or reduced beta-cell mass. Diabetes 59, 2178-2187.

Phillips, J. W., Clark, D. G., Henly, D. C., and Berry, M. N. (1995). The contribution of glucose cycling to the maintenance of steady-state levels of lactate by hepatocytes during glycolysis and gluconeogenesis. European journal of biochemistry/FEBS 227, 352-358.

Prentki, M., and Madiraju, S. R. (2008). Glycerolipid metabolism and signaling in health and disease. Endocr. Rev. 29, 647-676.

Prentki, M., and Madiraju, S. R. (2012). Glycerolipid/free fatty acid cycle and islet beta-cell function in health, obesity and diabetes. Mol. Cell. Endocrinol. 353, 88-100.

Puttmann, M., Krug, H., von Ochsenstein, E., and Kattermann, R. (1993). Fast HPLC determination of serum free fatty acids in the picomole range. Clinical chemistry 39, 825-832.

Qiang, L., Wang, L., Kon, N., Zhao, W., Lee, S., Zhang, Y., Rosenbaum, M., Zhao, Y., Gu, W., Farmer, S. R., et al. (2012). Brown remodeling of white adipose tissue by SirT1-dependent deacetylation of Ppargamma. Cell 150, 620-632.

Saddik, M., and Lopaschuk, G. D. (1991). Myocardial triglyceride turnover and contribution to energy substrate utilization in isolated working rat hearts. The Journal of biological chemistry 266, 8162-8170.

Segall, L., Lameloise, N., Assimacopoulos-Jeannet, F., Roche, E., Corkey, P., Thumelin, S., Corkey, B. E., and Prentki, M. (1999). Lipid rather than glucose metabolism is implicated in altered insulin secretion caused by oleate in INS-1 cells. The American journal of physiology 277, E521-528.

Zhao, S., Mugabo, Y., Iglesias, J., Xie, L., Delghingaro-Augusto, V., Lussier, R., Peyot, M. L., Joly, E., Taib, B., Davis, M. A., et al. (2014). alpha/beta-Hydrolase domain-6-accessible monoacylglycerol controls glucose-stimulated insulin secretion. Cell metabolism 19, 993-1007.

1. A method for increasing glycerol production and glycerol release from a mammalian cell, said method comprising contacting an effective amount of an agent capable of increasing the biological activity of a human glycerol-3-phosphate phosphatase (hG3PP) in the mammalian cell.

2. The method of claim 1, wherein the agent capable of increasing the biological activity of the hG3PP is succinic acid, a succinate salt and/or a nucleic acid expression system encoding the hG3PP.

3. The method of claim 1 or 2, wherein the mammalian cell is a mammalian pancreatic β cell or a mammalian hepatocyte.

4. The method of claim 1, wherein the mammalian cell is a mammalian cancerous cell.

5. The method of claim 1, wherein the mammalian cell is in vitro.

6. The method of claim 1, wherein the mammalian cell is located in a mammalian subject in need of increasing glycerol production and glycerol release from the mammalian cell and the method further comprises administering a therapeutically effective amount of the agent to the mammalian subject.

7. The method of claim 6, wherein the mammalian subject is afflicted by obesity, type II diabetes and/or metabolic syndrome X.

8. The method of claim 6, wherein the mammalian subject is afflicted by a cancer.

9.-16. (canceled)

17. A method for characterizing the usefulness of a test agent to increase glycerol production and glycerol release from a mammalian cell, said method comprising:
(a) providing a human glycerol-3-phosphate phosphatase (hG3PP) and a substrate of the human glycerol-3-phosphate phosphatase that can be cleaved by the hG3PP to generate at least one detectable moiety;
(b) combining the test agent with the hG3PP and the substrate under conditions so as to allow the cleavage of the substrate by the hG3PP and the generation of the at least one detectable moiety;
(c) determining a test amount of the at least one detectable moiety generated at step (b);
(d) comparing the test amount with a first control amount of the at least one detectable moiety, wherein the first control amount is derived from or obtained by combining the hG3PP and the substrate, in the absence of the test agent, under conditions so as to allow the cleavage of the substrate by the hG3PP and the generation of the at least one detectable moiety; and
(e) characterizing the test agent as being useful for increasing glycerol production and glycerol release from the mammalian cell when the test amount is determined to be higher than the first control amount.

18. The method of claim 17, wherein, at step (a), the substrate is provided at or near a saturating concentration.

19. The method of claim 17, wherein the substrate is glycerol-3-phosphate.

20. The method of claim 19, wherein the at least one detectable moiety is glycerol.

21. The method of claim 17, wherein the at least one detectable moiety is inorganic phosphate.

22. The method of claim 17, wherein step (a) further comprises providing a cellular extract of the mammalian cell comprising the hG3PP.

23. The method of claim 17, wherein step (a) further comprises providing the hG3PP in a substantially isolated form.

24. The method of claim 17, wherein step (a) further comprises providing the substrate in a substantially isolated form.

25. The method of claim 17, further comprising:
providing a second control amount obtained by combining the hG3PP, the substrate and succinic acid or a succinate salt under conditions so as to allow the cleavage of the substrate by the hG3PP and the generation of the at least one detectable moiety;
comparing the second control amount with the first control amount; and
characterizing the agent as being useful for increasing glycerol production and glycerol release from the mammalian cell from the mammalian cell only when the second control amount is higher than the first control amount.

26. The method of claim 17, wherein the mammalian cell is a mammalian pancreatic β cell.

27. (canceled)

28. The method of claim 17, wherein the mammalian cell is a mammalian hepatocyte.

29. (canceled)

30. The method of claim 17, wherein the mammalian cell is a cancerous cell.

31.-33. (canceled)


SEQUENCE LISTING (10 sequences)

SEQ ID NO: 1 - DNA, 21 bp, Artificial Sequence
  Oligonucleotide
aggcggacaucaucgggaatt

SEQ ID NO: 2 - DNA, 21 bp, Artificial Sequence
  Oligonucleotide
uucccgaugauguccgccugg

SEQ ID NO: 3 - DNA, 21 bp, Artificial Sequence
  Oligonucleotide
ctgagaaacggctaccacatc

SEQ ID NO: 4 - DNA, 21 bp, Artificial Sequence
  Oligonucleotide
ggcctcgaaagagtcctgtat

SEQ ID NO: 5 - DNA, 21 bp, Artificial Sequence
  Oligonucleotide
cttgctgcagacatggtcaac

SEQ ID NO: 6 - DNA, 21 bp, Artificial Sequence
  Oligonucleotide
gccattatggcgtgtgaagtc

SEQ ID NO: 7 - DNA, 21 bp, Artificial Sequence
  Oligonucleotide
ctggacacagacatcctcctg

SEQ ID NO: 8 - DNA, 22 bp, Artificial Sequence
  Oligonucleotide
tcactttcctgattgctcttca

SEQ ID NO: 9 - DNA, 21 bp, Artificial Sequence
  Oligonucleotide
gacaacctccactcactctgc

SEQ ID NO: 10 - DNA, 21 bp, Artificial Sequence
  Oligonucleotide
aagtttagctgggctgctgtt